(19) United States
(12) Patent Application Publication
(10) Pub. No.: US 2022/0406415 A1
(43) Pub. Date: Dec. 22, 2022

(54) SYSTEMS AND METHODS FOR SYNERGISTIC PESTICIDE SCREENING

(71) Applicant: Terramera, Inc., Vancouver (CA)

(72) Inventors: Costantinos LAMBRINOUDIS, Vancouver (CA); Sadegh SHOKATIAN, Vancouver (CA); Le Linh BUI, Vancouver (CA); Oliver SNOW, Squamish (CA)

(21) Appl. No.: 17/763,165

(22) PCT Filed: Sep. 25, 2020
(86) PCT No.: PCT/CA2020/051285

§ 371 Date: Mar. 23, 2022

Related U.S. Application Data
(60) Provisional application No. 62/906,341, filed on Sep. 26, 2019.
(60) Provisional application No. 62/987,751, filed on Mar. 10, 2020.

(51) Int. Cl. G16C 20/30; G16C 20/70; G16C 20/64

(52) U.S. Cl. CPC G16C 20/30; G16C 20/70; G16C 20/64

(57) ABSTRACT

A computer system that predicts synergistic interactions between pesticidal and synergistic compounds of a pesticidal composition is described. The system provides a trained classifier that provides probabilistic predictions of the synergy between two or more compounds on a pest. The system may select features for transformation, encode them, generate one or more predictions, and combine the predictions. The predictions may be evaluated by experimental testing, e.g. in vitro or in planta, and/or used to formulate and/or apply a pesticidal composition.

### REFERENCE TO RELATED APPLICATIONS

This application claims priority to and the benefit of U.S. Provisional Patent Application No. 62/906,341 filed on 26 Sep. 2019 and U.S. Provisional Patent Application No. 62/987,751 filed 10 Mar. 2020, the disclosures of which are incorporated herein by reference in their entirety.

### TECHNICAL FIELD

The present disclosure relates generally to pesticidal compositions and particularly to pesticidal compositions with other active or formulation relevant ingredients.

### BACKGROUND

Pesticides (e.g. fungicides, herbicides, nematicides, insecticides, bactericides, rodenticides, virucides, miticides, algicides, molluscicides) are compositions used in domestic, agricultural, industrial and commercial settings. Pesticides are used to control and/or suppress unwanted pests which, if not controlled, can harm plants (such as crops), animals, humans, and/or other organisms. Accordingly, there is a need for efficacious pesticidal compositions.

There is also a desire to reduce the quantity in which pesticides are used, whether to avoid deleterious environmental effects, to reduce costs, or for other reasons. For example, chemical pesticides are often used in agricultural settings, where a variety of plant pests, such as insects, worms, nematodes, fungi, and plant pathogens such as viruses and bacteria, are known to cause significant damage to seeds, ornamental plants, and crop plants. Such compositions are often expensive, potentially toxic (e.g. to humans, animals, and/or the environment), contributory to growing pesticidal resistance among pest organisms, subject to regulatory restrictions, and/or long-lasting after application. It is typically beneficial to farmers, consumers and the surrounding environment to use the least amount of chemical pesticides possible, while continuing to control pest growth in order to maximize crop yield.

Natural or biologically-derived pesticidal compositions have been proposed for use in place of some chemical pesticides in response to such concerns. However, some natural or biologically-derived pesticides have proven less efficacious or consistent in their performance in comparison with competing chemical pesticides, leading to limited adoption.

There is a general desire for improved pesticides and pesticidal compositions to allow for effective, economical and environmentally safe control of undesirable pests (such as insect, plant, fungal, nematode, mollusk, mite, rodent, viral and bacterial pests). In particular, there remains a need for pesticidal compositions that reduce the quantity of pesticidal agents and/or pesticidal active ingredients required to obtain desired or acceptable levels of control of pests in use.

Identifying improved pesticidal compositions is generally challenging. Synergistic pesticidal compositions, wherein the quantity of a pesticidal active ingredient is reduced via synergistic efficacy with some synergistic additive, are very rare. For example, a systematic screening of about 120,000 two-component combinations based on reference-listed compounds found only 5% of two-component pairs including fluconazole, a triazole fungicidal compound related to certain azole agricultural fungicide compounds, were synergistic (c.f. Borisy et al., Systematic discovery of multicomponent therapeutics. Proc. Natl Acad. Sci. 100:7977-7982 (2003)). Screening the more than 10\hat{ }60 possible compositions for potential synergistic efficacy in a particular use is infeasible with conventional experimental techniques—for instance, a laboratory of 10 chemists might screen on the order of 10\hat{ }4-10\hat{ }6 such compositions in a year.

There is thus a general desire for improved systems and methods for screening pesticidal compositions for synergistic efficacy.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a computing system comprising one or more processors and a memory containing instructions which cause the one or more processors to perform a method, and/or a non-transitory machine-readable medium storing such instructions. The method is for generating a prediction of a synergistic interaction between two or more compounds against one or more pests. The method comprises receiving a first representation of a pesticidal compound; receiving a second representation of a synergistic compound; identifying, based on the first representation, a first chemical feature of the pesticidal compound; identifying, based on the second representation, a second chemical feature of the synergistic compound; generating an encoded representation of a composition comprising the pesticidal and synergistic compounds by encoding the first and second chemical features; and generating one or more predictions of a synergistic interaction between the pesticidal compound and the synergistic compound against one or more pests, said generating comprising: transforming the encoded representation based on trained parameters of a classifier, the trained parameters of the classifier having been trained over at least one synergistic interaction between compounds of at least one composition against at least one of the one or more pests.

In some embodiments, wherein the one or more predictions of synergistic interaction comprise a plurality of predictions and the method further comprises: combining the plurality of synergy predictions into a combined synergy. In some implementations, the method further comprises determining at least one of: a confidence interval, a standard deviation, and a variance based on the plurality of predictions. In some implementations, the classifier comprises a stochastic classifier and generating the one or more predictions comprises transforming the encoded representation based on the trained parameters of the classifier over a plurality of iterations and generating a prediction for each iteration.

In some embodiments, generating the encoded representation comprises generating a first encoded compound representation based on the first chemical feature of the pesticidal compound and generating a second encoded compound representation based on the second chemical feature of the synergistic compound and wherein generating the one or more predictions comprises generating the one or more predictions based on the first and second encoded compound representations.

In some embodiments, wherein generating the encoded representation comprises generating the encoded representation to be lower-dimensional than the encodable representation.

In some embodiments, wherein the generating the encoded representation comprises transforming an encodable representation of at least one of the pesticidal compound and the synergistic compound into the encoded representation based on trained parameters of an encoder model. In some implementations, the encoder model comprises an encoder portion of a variational autoencoder, the encoder portion operable to transform the encodable representation from an input space to a latent space of the variational autoencoder. In some implementations, the trained parameters of the encoder model have been trained over a different training set than the trained parameters of the classifier.

In some embodiments, the method further comprises selecting the classifier from a plurality of classifiers based on the one or more pests. In some implementations, the method further comprises receiving a representation of the one or more pests and selecting the classifier comprises selecting the classifier based on the representation of the one or more pests. In some implementations, the classifier is a first one of a plurality of classifiers, at least a second classifier of the plurality having been trained against different pests than the one or more pests, and selecting the classifier from the plurality of classifiers comprises selecting one of the first and second classifiers based on the one or more pests. In some implementations, the classifier comprises an ensemble classifier comprising a plurality of constituent classifiers, the plurality of constituent classifiers comprising at least a first constituent classifier and a second constituent classifier, respective trained parameters of the first and second constituent classifiers each having been trained over at least one synergistic interaction between compounds of at least one composition against at least one of the one or more pests. In some implementations, generating one or more predictions comprises generating a first prediction based on the first constituent classifier and generating a second prediction based on the second constituent classifier.

In some embodiments, generating an enhanced representation at least one of the pesticidal and synergistic compound, the enhanced representation comprising an enhanced chemical feature comprising at least one of the first and second chemical features. In some implementations, generating the enhanced representation comprises determining the enhanced chemical feature based on trained parameters of a quantitative structure-activity relationship model.

In some embodiments, receiving a third representation of a third compound and excluding an excluded composition comprising the third compound from prediction based on determining at least one of: a chemical feature of the third compound matches an exclusion rule, an availability value corresponding to the third compound being less than a threshold, a similarity metric between the third compound and a fourth compound being greater than a threshold, and a toxicity indication of the third compound matches a toxicity criterion.

In some embodiments, the pesticidal compound is selected from the group consisting of: fungicides, herbicides, nematicides, insecticides, bactericides, rodenticides, virucides, miticides, and molluscicides.

In some embodiments, the method comprises selecting at least one of the first and second chemical features from the group consisting of: representations of aromaticity, representations of electronegativity, representations of polarity, representations of hydrophilicity/hydrophobicity, and representations of hybridizations of at least one of the pesticidal and synergistic compounds.

In some embodiments, the one or more pests comprise the at least one training pest. In some embodiments, the at least one training pest shares a pesticidal mode of action with at least one of the one or more pests, without necessarily being included in the one or more pests.

In some embodiments, the trained parameters of the classifier have been trained by: determining an importance metric for each of a plurality of training compositions; selecting one or more high-importance compositions from the plurality of training compositions based on the importance metric for each of the one or more high-importance compositions; and updating the trained parameters of the classified based on the one or more high-important compositions. In some embodiments, determining the importance metric for a given composition comprises determining the importance metric for the given training composition based on a variance of one or more training predictions of the synergistic interaction between a pesticidal compound of the training composition and a synergistic compound of the training composition.

In some embodiments, selecting one or more high-importance compositions comprises selecting the one or more high-importance compositions based on a representativeness criterion. In some embodiments, selecting the one or more high-importance compositions based on a representativeness criterion comprises determining a plurality of clusters of the plurality of training compositions and selecting at least one high-importance compositions from each of at least two of the plurality of clusters. In some embodiments, determining the plurality of clusters of the plurality of training compositions comprises determining a graph similarity metric between at least one graph representing at least one compound of a first one of the training compositions and at least one graph representing at least one compound of a second one of the training compositions.

In some embodiments, the prediction of synergistic interaction is verified or evaluated by combining the relevant pesticidal compound and synergistic compound to yield a composition and exposing one or more pests to the composition in a test environment. In some embodiments, the prediction of synergistic interaction is used to formulate a pesticidal composition by formulating the pesticidal compound to contain the relevant pesticidal compound and synergistic compound. In some embodiments, the prediction of synergistic interaction is used to manufacture a pesticidal composition by mixing the relevant pesticidal compound and synergistic compound together with any desired formulation components or additives to yield the pesticidal composition. In some embodiments, the prediction of synergistic interaction is used to treat one or more pests affecting a non-target organism by exposing the non-target organism to a pesticidal composition containing the pesticidal compound and the synergistic compound. In some embodiments, to treat one or more pests affecting a non-target organism, a plurality of predictions of synergistic interaction are determined and evaluated to select a combination of one of a plurality of pesticidal compounds and a corresponding one of a plurality of synergistic compounds. The non-target organism is then exposed to a composition containing the selected combination of the one of the plurality of pesticidal compounds and the corresponding one of the plurality of synergistic compounds.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 illustrates schematically an example system for predicting synergistic and/or antagonistic interactions between two or more compounds of a candidate pesticidal composition upon at least one pest.

FIG. 2 is a flow chart of an example method for generating predictions of synergistic and/or antagonistic interactions between two or more compounds of a candidate pesticidal composition upon at least one pest by the system of FIG. 1.

FIG. 3 is a flow chart of an example method for screening candidate pesticidal compositions by an example selector of the system of FIG. 1.

FIG. 4 is a flow chart of an example method for encoding candidate pesticidal compositions by an example encoder of the system of FIG. 1.

FIG. 5 is a flow chart of an example method for generating one or more predictions of synergistic and/or antagonistic interactions between compounds of a candidate pesticidal composition by an example classifier of the system of FIG. 1.

FIG. 6 is a flow chart of an example method for training parameters of an example classifier of the system of FIG. 1.

FIG. 7 illustrates schematically an example data flow for an example combiner of the system of FIG. 1.

FIG. 8 illustrates an exemplary computer system adapted to provide the system of FIG. 1.

FIG. 9 illustrates an exemplary method of evaluating the efficacy of a pesticidal composition prepared using a prediction of synergistic interaction.

FIG. 10 illustrates an exemplary method of formulating a pesticidal composition using a prediction of synergistic interaction.

FIG. 11 illustrates an exemplary method of manufacturing a pesticidal composition using a prediction of synergistic interactions for a plurality of candidate pesticidal compositions.

FIG. 12 illustrates a method of treating one or more pests affecting a non-target organism using a prediction of synergistic interaction.

FIG. 13 illustrates a method of treating one or more pests affecting a non-target organism using a prediction of synergistic interactions for a plurality of candidate pesticidal compositions.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

### Overview

Conventional methods of determining synergistic (and/or antagonistic) interactions between pesticidal and other compounds generally involve a series of lab screening and field trial experiments. Initial plate tests at the lab screening phase often find that there is no synergistic interaction. Subsequent testing is often in planta and can consume considerable resources; for instance, in an agricultural context, such testing can last several growing seasons, involve several staff and considerable growing space and infrastructure, and may require repetition to mitigate systemic error and/or to respond to specific issues that arise during testing.

The present disclosure provides systems and methods for screening candidate pesticidal compositions of two or more compounds for synergistic interactions against one or more pests. The described system and methods can, in certain circumstances, efficiently and accurately predict which candidate pesticidal compositions are likely to have a synergistic interaction against one or more pests. The described system and methods may be used in addition to (e.g. prior to and/or in parallel with) or even instead of conventional laboratory-based screening. Subsequent testing of compositions predicted to be likely to lack the desired synergistic interaction may be reduced or eliminated, thereby potentially accelerating the discovery of synergistic pesticidal compositions.

The systems and methods described herein predict synergistic interactions (or lack thereof) against at least one pest in compositions comprising at least one pesticidal active ingredient and at least one synergistic compound. (“Synergistic compound” as used herein does not require that the compound in fact be synergistic, but rather refers to the fact that the compound is assessed for synergistic interactions with the pesticidal active ingredient.) A synergistic pesticidal composition screening system may be configured to operate in a number of different operating modes, depending upon the desired use. In some embodiments, a synergistic pesticidal composition screening system generates predictions related to the probability of whether a synergistic interaction is likely for a candidate pesticidal composition. Such predictions may enable a user to select candidate pesticidal compositions which are likely to possess a synergistic interaction for further testing steps (e.g. to confirm the predicted synergistic interaction) based on the prediction.

In some embodiments, a synergistic pesticidal composition screening system generates predictions of the degree of synergistic interaction (if any) exhibited by a candidate pesticidal composition. Such predictions may enable a user to select candidate pesticidal compositions which are most likely to exhibit synergistic interaction, or which are likely to exhibit at least a certain degree of synergistic interaction, for further testing based on the prediction.

In some embodiments, a synergistic pesticidal composition screening system predicts a synergy metric describing a synergistic interaction exhibited by a candidate pesticidal composition. Any suitable synergy metric may be predicted; for example, the system may predict a minimum inhibitory concentration (MIC) and/or fractional inhibitory concentration index (FICI) value for the candidate pesticidal composition. The system may alternatively, or in addition, predict any of the various other synergy metrics available, including, e.g., those described by Greco et al., The search for synergy: a critical review from a response surface perspective, Pharmacological Reviews 47, 331-85, incorporated herein by reference.

In some embodiments, a synergistic pesticidal composition screening system predicts a metric of improved pesticidal effectiveness of a candidate pesticidal composition upon one or more pest organisms. The predicted metric may be used to predict the quantity of candidate pesticidal composition required for pesticidal effectiveness in the field. Such predictions may enable a user to screen candidate pesticidal compositions based on such predicted quantities. For example, the predicted quantity may be combined with an estimated per-unit cost of the candidate pesticidal composition (e.g. by multiplication) to determine a predicted cost per unit of efficacy. Candidate pesticidal compositions may be screened, ranked, presented to a user, or otherwise output based on such predicted quantity and/or predicted cost per unit of efficacy.

One or more of the foregoing embodiments may be provided as modes of operation of a synergistic pesticidal composition screening system. As described in greater detail below, the synergistic pesticidal composition screening system generates predictions based on trained parameters. In some embodiments, the trained parameters may be further trained based on results of laboratory and/or field tests performed after the system generates predictions.

The foregoing overview refers generally to synergistic interactions. Antagonistic interactions may also, or alternatively, be predicted. Except where the context requires otherwise, the present disclosure applies equally to synergistic and antagonistic interactions.

These and other aspects and advantages will become apparent when the description below is read in conjunction with the accompanying drawings.

### Definitions

As used in this specification the following definitions are used:

Candidate pesticidal composition: a combination of at least two candidate compounds, including at least one pesticidal compound and at least one potentially synergistic and/or antagonistic compound (referred to generally herein for convenience as a synergistic compound), with or without a defined mixing ratio, and optionally comprising one or more additional compounds. The candidate pesticidal composition may comprise a mixture.

Non-target organism: a non-target organism is an organism upon which pests have a harmful effect. Non-target organisms may include plants, animals, and any other effected organism, and in particular include crop plants and crop animals such as domesticated farm animals. For example, non-target organisms include (without limitation) crop plants such as cucumbers and soybean plants and crop animals such as pigs and cattle.

Pest: an undesirable organism living in an environment, often having harmful effects on one or more host organisms in the environment (e.g. crop plants). Pests can be insects, plants, fungi, nematodes, mollusks, mites, rodents, viruses, bacteria, and/or other organisms. An example of a pest is powdery mildew, which grows on (and harms) a variety of crop plants, such as soybean plants.

MIC: the Minimum Inhibitory Concentration is the lowest concentration of a chemical which prevents growth of a pest.

FICI: Fractional Inhibitory Concentration Index: a metric of synergy. Indicates degree of ‘synergy’ (FICI<0.5), ‘antagonism’ (FICI>4.0) and ‘no interaction’ (FICI>0.5-4.0).

Metric: a system of standard for measurement. A metric value is a distinct value within a specified system of measurement. An example of a metric is FICI, and a calculated FICI score is a metric value. A metric need not be produced from measurement directly, and may be predicted (e.g. as described herein with reference to the synergistic pesticidal composition screening system predicting a metric value).

Synergistic interaction: an effect of two or more chemical compounds taken together which is greater than the sum of their separate effects at the same doses. Compositions comprising two or more compounds which possess a synergistic interaction are said to have synergy.

Antagonistic interaction: an effect of two or more chemical compounds taken together which is less than the sum of their separate effects at the same doses. Compositions comprising two or more compounds which possess an antagonistic interaction are said to have antagonism.

Active ingredient: one or more chemical compounds (e.g. a molecule, a complex, a mixture, etc.) that has the effect of inhibiting, stimulating or otherwise altering the production or biological activity of at least one pest. Compounds of an active ingredient are sometimes referred to as “active compounds”.

Pesticide: a substance that is effective for inhibiting the growth and/or biological activity of one or more pests.

All other words have their normal meanings when used in the field of chemistry and biochemistry.

### Overview of a Synergistic Pesticidal Composition Screening System and Method

The present disclosure provides a synergistic pesticidal composition screening system and methods of its operation. In some embodiments, the synergistic pesticidal composition screening system predicts a probability that two or more candidate compounds exhibit one or more synergistic (and/or antagonistic) interactions. In some embodiments, the synergistic pesticidal composition screening system predicts a degree of synergistic (and/or antagonistic) interactions between candidate compounds. In some embodiments, the synergistic pesticidal composition screening system predicts a metric value that describes the synergistic (and/or antagonistic) interactions, such as a MIC and/or FICI value, of a candidate pesticidal composition. The synergistic pesticidal composition screening system generates a prediction by transforming a digital representation of the candidate compounds based on a set of trained parameters as described in greater detail herein. The prediction(s) generated by the system may be used, for example, in an industrial chemical composition screening process to predict whether a candidate pesticidal composition is likely to have a synergistic (and/or antagonistic) interaction, and optionally the degree of that interaction (e.g. strong/weak) and/or a metric value describing that interaction (e.g. a MIC and/or FICI value, a quantity of composition required to obtain a certain degree of efficacy, etc.).

Active ingredients of pesticidal compositions (and thus pesticidal compositions themselves) often have limited lifetimes. Pests can evolve resistance to the mode of action of the active ingredient, thus making a pesticidal composition less effective or ineffective over time. For example, certain pests (e.g. insects, nematodes, fungi, yeasts, rusts) have evolved resistance to the chemical compounds that have been used to manage their presence in crop fields. As pests evolve resistance, commercial pesticides require new active ingredients to manage them. The synergistic pesticidal composition screening system attempts to identify, by its predictions, previously-unknown synergistic interactions between compounds, thereby identifying candidate pesticidal compositions of those compounds that are relatively more likely to have greater efficacy against resistant organisms (relative to compositions not identified by the system as possessing synergistic interactions). In certain circumstances, active ingredients which were previously rendered less effective or ineffective (e.g. due to increased resistance) can be made effective again by combination with candidate compounds predicted by the system to possess a synergistic interaction with the active ingredient. The presently-described synergistic pesticidal composition screening system can thus make the identification of new pesticidal compositions in a computationally-tractable way.

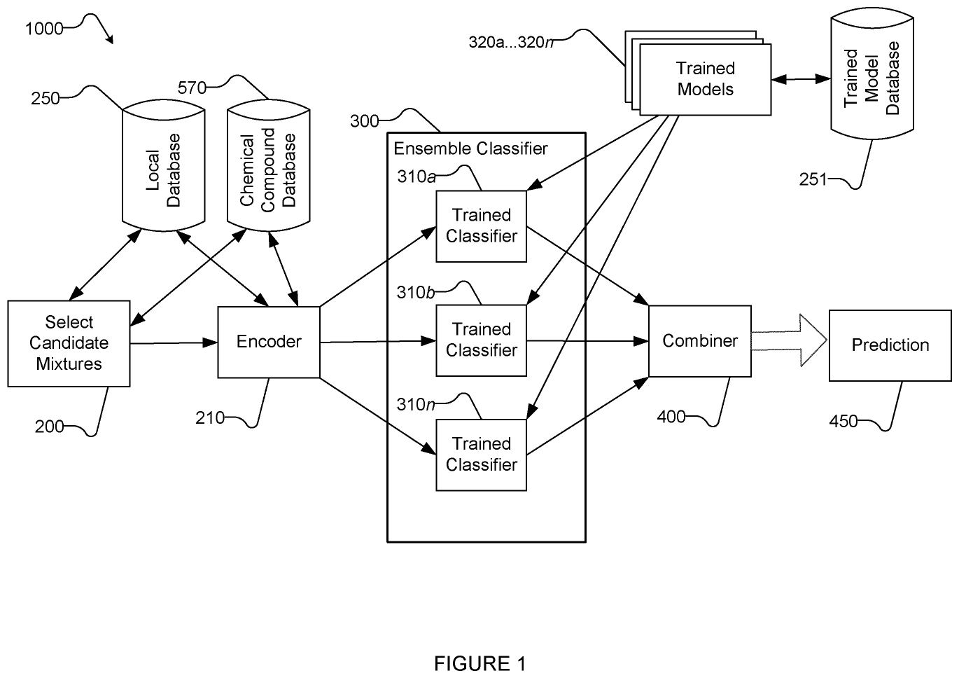

FIG. 1 illustrates an example synergistic pesticidal composition screening system 1000, which in a first exemplary embodiment comprises a computer system for predicting characteristics of synergistic and/or antagonistic interactions (e.g. existence, degree, and/or an associated metric value) between two or more compounds on at least one pest. System 1000 and its methods of operation are described herein.

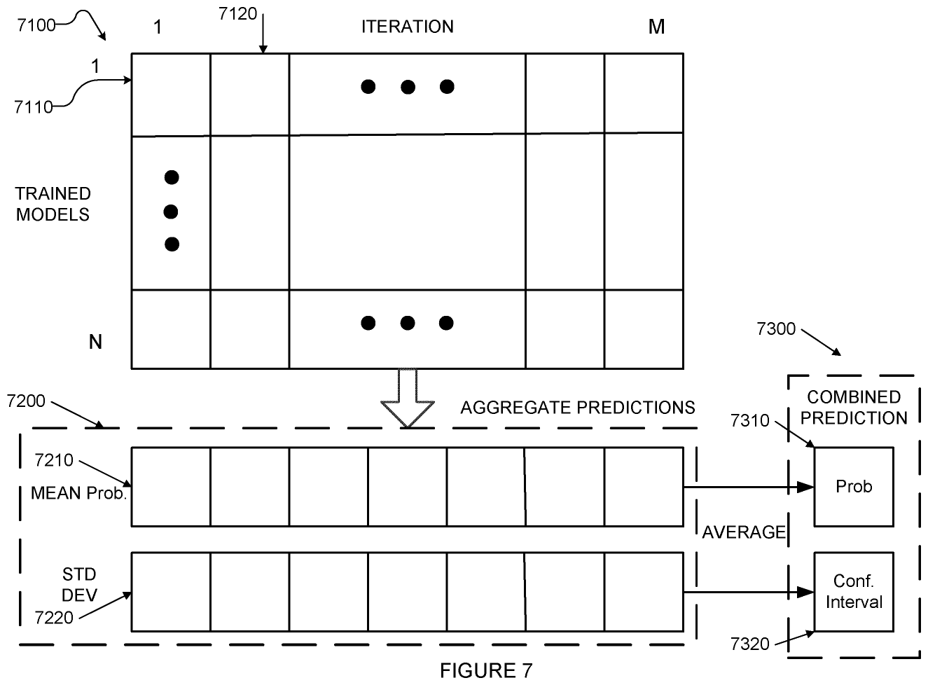

System 1000 is a computer system providing a selector 200, encoder 210, ensemble classifier 300, and combiner 400. System 1000 is optionally in communication with one or more datastores, such as databases 250, 251, 570. Selector 200, encoder 210, ensemble classifier 300, and combiner 400 may be provided by hardware and/or software and are referred to generally herein as “modules” of system 1000. At a high level, selector 200 receives digital representations of one or more candidate pesticidal compositions and selects one or more selected candidate pesticidal compositions (e.g. according to method 3000, described elsewhere herein). Encoder 210 receives the one or more selected candidate pesticidal compositions and, for each selected candidate pesticidal composition, generates an encoded representation of the selected candidate pesticidal composition for classification by classifier 300 (e.g. according to method 4000, described elsewhere herein). Classifier 300 receives each encoded representation and generates one or more predictions for each encoded representation based on one or more sets of trained parameters (e.g. according to method 5000, described elsewhere herein). In some embodiments, including the depicted embodiment, classifier 300 comprises an ensemble classifier comprising a plurality of trained classifiers 310a . . . 310n, each of which generates a prediction. In at least some embodiments where classifier 300 generates a plurality of predictions for a selected candidate pesticidal composition, combiner 400 receives the plurality of predictions and generates a combined prediction 450 based on the plurality of predictions (e.g. as described in greater detail with reference to FIG. 7).

System 1000 can be trained to predict any of a variety of interactions between compounds of a candidate pesticidal composition. In some implementations, system 1000 generates prediction 450 by predicting a predicted probability of existence of a synergistic (and/or antagonistic) interaction between compounds of the candidate pesticidal composition and at least one pest, a predicted degree of such interaction, and/or a predicted metric value describing such interaction. In some embodiments, system 1000 additionally or alternatively generates prediction 450 by predicting toxicity of the candidate pesticidal composition against at least one organism (e.g. the at least one pest, at least one crop, etc.). In some embodiments, system 1000 generates prediction 450 by determining one or more metrics and/or other attributes derived from a predicted synergistic and/or antagonistic interaction between compounds of the candidate pesticidal composition and/or the at least one pest, such as predicting mitigation of resistance by one or more pests of the at least one pest, predicted effectiveness of the candidate pesticidal composition, and/or predicted composition formula (e.g. expressed as compound ratios).

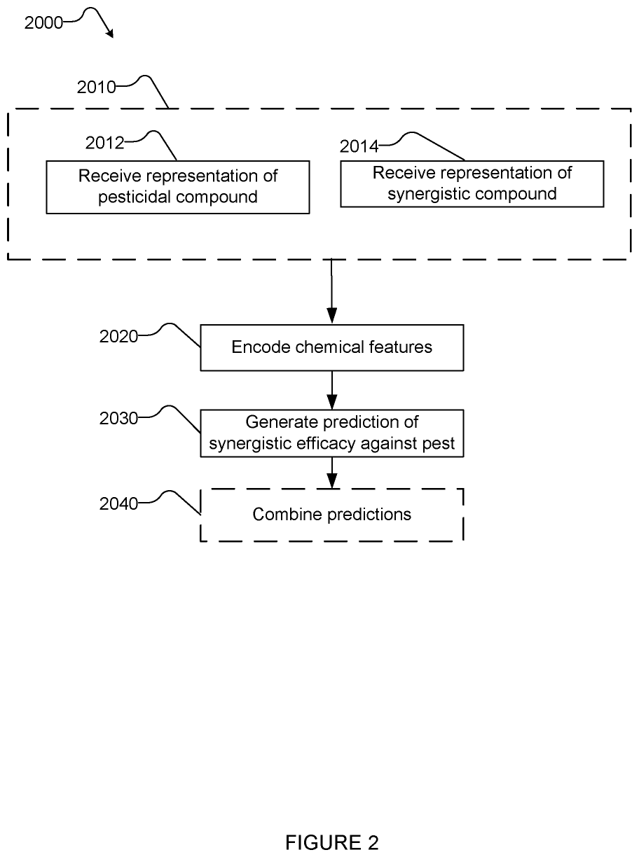

FIG. 2 shows an example method 2000 for generating predictions of synergistic and/or antagonistic interactions between two or more compounds of a candidate pesticidal composition. The method is performed by a computer system (e.g. system 1000). At 2010, the computer system receives a representation of the candidate pesticidal composition. Act 2010 may be performed, for example, by selector 200 of system 1000 and may comprise any of the acts described below with reference to method 3000, such as enhancing representations of the composition and/or constituent compounds, filtering compositions, feature selection, and so on. In some embodiments, act 2010 comprises receiving a representation of a pesticidal compound (at 2012) and receiving a representation of a synergistic compound (at 2014). In some embodiments, act 2010 comprises receiving a representation of one or more pests which the candidate pesticidal composition is to be assessed for synergistic pesticidal efficacy against. In some embodiments, act 2010 also or alternatively comprises receiving mixture information, such as mixture ratios and/or mixture ratio ranges.

At 2020, the computer system generates an encoded representation of the candidate pesticidal composition for classification by classifier 300 by encoding chemical features of the pesticidal and synergistic compounds based on the representation(s) received at 2010. Act 2020 may be performed, for example, by encoder 210 and/or classifier 300 of system 1000 (which may optionally be provided by one machine learning model) and may comprise any of the acts described below with reference to method 4000, such as compression, feature selection, and/or transcoding (e.g. to a latent space defined by encoder 210 and/or classifier 300). Act 2030 comprises transforming each raw representation into an encoded representation of the candidate pesticidal composition (which may comprise a unitary representation, such as a single latent vector for the composition, and/or a plurality of representations, such as one for each compound of the candidate pesticidal composition).

At 2030, the computer system generates a prediction of synergistic efficacy of the candidate pesticidal composition against one or more pests based on the encoded representation generated at 2020 and on trained parameters of a classifier model. Act 2030 may be performed, for example, by classifier 300 of system 1000 (e.g. trained in accordance with method 6000) and may comprise any of the acts described below with reference to method 5000. In at least some embodiments, act 2030 comprises transforming the encoded representation based on trained parameters of the classifier, the trained parameters of the classifier having been trained over at least one synergistic interaction between compounds of at least one composition against at least one of the one or more pests. Act 2030 may comprise generating a plurality of predictions, e.g. via a stochastic classifier, as described in greater detail elsewhere herein.

At 2040, the computer system optionally combines a plurality of predictions to generate a combined prediction (e.g. prediction 450). Act 2040 may be performed, for example, by combiner 400 of system 1000 and may comprise any of the acts described below with reference to combiner 400 and the data flow diagram of FIG. 7. In some embodiments, act 2040 comprises generating a confidence measure (e.g. a confidence interval) for the combined prediction, as described in greater detail elsewhere herein.

### Selecting Candidate Pesticidal Compositions

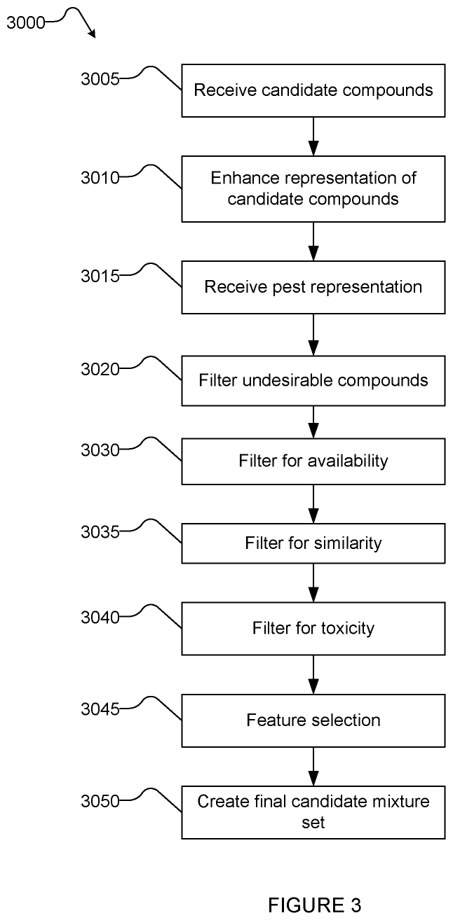

In at least some embodiments, the operation of system 1000 begins with selector 200. FIG. 3 is a flow chart of an example method 3000 for selecting candidate pesticidal compositions by system 1000. Method 3000 may be performed in whole or in part by selector 200 of system 1000. Method 3000 selects candidate pesticidal compositions for system 1000 to evaluate for synergistic potential. Since many candidate pesticidal compositions will generally be available, in at least some implementations method 3000 comprises removing from consideration certain compounds and/or compositions from further evaluation.

At 3005, system 1000 (e.g. by selector 200) receives at least a partial digital representation of each of one or more compounds. The one or more compounds may be provided by a user, by another computing system, retrieved from a datastore, and/or otherwise obtained via any suitable technique. Each digital representation comprises a representation of the compounds' chemical structure and/or the compounds' chemical properties (which may include, for example, the compounds' known effects upon classes of organisms, such as pests, crop plants, etc.). The one or more compounds may comprise natural and/or synthetic compounds. System 1000 may optionally also receive a representation of at least one pest. In some embodiments, system 1000 also receives candidate pesticidal composition formulation parameters, such as compositional ratios and/or constituent percentages of at least one of the compounds in the candidate pesticidal composition. The various representations and parameters received by system 1000 are referred to collectively herein as the received representation of the candidate pesticidal composition.

In some embodiments, system 1000 receives a representation of one compound of the candidate pesticidal composition at 3005, for example in embodiments where classifier 300 and/or encoder 210 are trained over synergistic compounds' synergistic interactions with the pesticidal compound, in which case the pesticidal compound may be implicitly represented by trained classifier 300 and/or encoder 210 without necessarily requiring receipt of an explicit representation of the pesticidal compound. In some embodiments, the pesticidal compound is predetermined and its representation is made available to system 1000 at the time method 3000 commences; accessing a predetermined representation during method 3000 is included within the meaning of “receiving” such representation.

Optionally, at 3010 system 1000 enhances the received representation with additional chemical properties to produce an enhanced representation. For example, selector 200 may obtain from a datastore (such as a local memory, database 250, database 570, or other suitable datastore) descriptions of the plurality of compounds' atomic and molecular information (e.g. molecular structure, molecular weight, constituent atoms, bonding type (e.g. single, double, triple, aromatic)), atomic information (e.g. atomic number, hybridization, aromatic ring member, implicit and explicit valence, degree (number of bonds)) and/or other chemical properties (e.g. functional group in specific location, charge distribution). In some embodiments, system 1000 comprises a trained model for generating additional chemical properties (e.g. as part of selector 200) and enhances the received representation by generating such additional chemical properties based on trained parameters of the trained model. For example, system 1000 may comprise a quantitative structure—activity relationship (QSAR) model, and 3005 may comprise generating one or more properties by the QSAR model and adding at least one of the one or more properties to the enhanced representation.

In some embodiments, the at least a partial digital representation of the compounds of the candidate pesticidal composition may comprise an identification of a composition or a class of compounds (thus permitting indirect identification of compounds). In some implementations, if the candidate pesticidal composition comprises a composition for which additional information is available to system 1000 (e.g. in an accessible datastore), system 1000 (e.g. at selector 200) enhances the received representation by retrieving at least a portion of that additional information and adding the retrieved information to the enhanced representation. In some embodiments such additional information comprises the chemical constituents and/or ratios of the composition. For example, selector 200 may add the constituent compounds and, optionally, their associated concentrations to the enhanced representation of the candidate pesticidal composition. Chemical composition information may be stored in a reference chemical database (e.g. database 250 and/or database 570 of FIG. 1). System 1000 may add such constituent compounds to the candidate pesticidal composition.

In some implementations, if the at least a partial representation received by system 1000 comprises one or more identifiers identifying one or more classes of compounds as an ingredient of the candidate pesticidal composition, system 1000 may (e.g. by selector 200) generate a plurality of candidate pesticidal compositions based on the one or more classes of compounds. For example, selector 200 may determine (e.g. based on information in a datastore such as database 250 and/or database 570), for each identified class of compounds, a set of compounds in that class. Selector 200 may generate a plurality of candidate pesticidal compositions by generating a plurality of enhanced representations, each enhanced representation comprising a different one of the compounds in the identified class. (In cases where multiple ingredients are identified in this way, each enhanced representation would comprise a different combination of compounds from the respective classes; a given compound could be repeated between representations by virtue of permutation.)

In some implementations, if a candidate pesticidal composition is selected that has a plurality of formulations (as may be the case, for example, with natural compositions such as extracts), system 1000 (e.g. by selector 200) may select one or more such formulations. For example, selector 200 may generate a plurality of enhanced representations of the candidate pesticidal composition, each corresponding to a different one of the formulations. Selector 200 may select the one or more formulations in any appropriate way, including: selecting all of the available formulations, selecting each formulation that satisfies a rule (e.g. selecting the formulation with the lowest complexity according to a complexity metric, least environmental impact based on an environmental metric, lowest cost based on cost information associated with each formulation, etc.), selecting a plurality of formulations with the highest rank according to a ranking algorithm, selecting one or more formulations psuedorandomly, requesting a selection from a user, and/or otherwise selecting the one or more formulations in any appropriate way. In some embodiments, system 1000 determines an average mixture ratio (e.g. via arithmetic mean, mode, or other suitable measure) based on the available formulations and adds that average mixture ratio to the enhanced representation of the candidate pesticidal composition.

In some embodiments, if the candidate pesticidal composition comprises a compound with more than one isomer, system 1000 (e.g. by selector 200) may select an isomer in any appropriate way, including any of the selection techniques described above with respect to formulations. If more than one isomer is selected, the system 1000 may generate a plurality of enhanced representations of the candidate pesticidal composition, each corresponding to a different one of the isomers.

In some embodiments, 3010 comprises receiving mixture ratios and/or mixture ratio ranges for one or more compounds (and/or constituent composition (s) and/or compound classes, as appropriate) that are to be included in the candidate pesticidal composition(s). If system 1000 receives a mixture ratio range, system 1000 may (e.g. by selector 200) select one or more mixture ratios within the mixture ratio range and generate a plurality of enhanced representations of the candidate pesticidal composition, each corresponding to a different one of the mixture ratios. System 1000 may generate such mixture ratios, for example, based on a predetermined parameter (e.g. system 1000 may generate n mixture ratios for some parameter n, the ratios spaced evenly apart within the range and including the extrema), a user selection, and/or any other suitable selection.

In some embodiments, 3010 comprises determining one or more fingerprint(s) for each candidate compound. The enhanced representation generated by system 1000 may in such embodiments comprise one or more fingerprints. In some embodiments, a fingerprint for a compound comprises a combination of a graph representation of the compound, combined with additional properties for the candidate compound (e.g. the various properties described above). The graph representation of each compound represents the structure of the compound molecule, with nodes of the graph for each atom in the molecule and the bonds represented as graph edges. System 1000 may further enhance the graph representation for each node (atom) in the compound with atomic properties such as atomic number, hybridization, whether the atom is part of an aromatic ring structure, implicit valence, and/or degree of its bonds. System 1000 may additionally or alternatively enhance the graph representation for each graph edge (bond) with properties such as the type of bond (e.g. single, double, triple, aromatic).

In various embodiments, different types of fingerprints that can be used, including a normalized Coulomb matrix (Rupp et. al), “bag of bonds” (Hansen, et al.), and other fingerprinting algorithms, such as those provided by RDKit such as Atom-Pair, Topological Torsion, Extended Connectivity Footprint (ECFP), E-state fingerprints, Avalon fingerprints, ErG, Morgan, MACCS. In some embodiments, system 1000 determines a plurality of fingerprints (e.g. for use in similarity screening at 3035, as described in greater detail elsewhere herein). In at least one implementation, system 1000 determines a Morgan and MACCS fingerprint for each candidate compound and adds both such fingerprints to the enhanced representation.

At 3015, system 1000 optionally obtains, for each of one or more pests, a representation of the pest. The representation may comprise, for example, an identifier of the pest (such as a name, an index, and/or a categorial variable) and/or a representation of at least a portion of the pest's genome. System 1000 may add the representations of the one or more pests (and/or information derived therefrom—e.g. an index may be derived from a name of a pest received by system 1000) to the enhanced representation of the composition and/or otherwise associate the representations of the one or more pests (and/or information derived therefrom) with the enhanced representation of the composition. The representations of the one or more pests may be predefined, received from a user, received from a datastore and/or another computer system, and/or otherwise received by system 1000.

In some embodiments, system 1000 alternatively or additionally receives, for each of one or more non-target organisms, representations of the non-target organism. A non-target organism may comprise, for example, a host plant, animal, or other organism on which a pest feeds, resides, or is otherwise proximate to during application of the pesticidal composition. The representation may comprise, for example, an identifier of the non-target organism (such as a name, an index, and/or a categorial variable) and/or a representation of at least a portion of the non-target organism's genome. System 1000 may add the representations of the one or more non-target organisms (and/or information derived therefrom—e.g. an index may be derived from a name of a non-target organism received by system 1000) to the enhanced representation of the composition and/or otherwise associate the representations of the one or more non-target organisms (and/or information derived therefrom) with the enhanced representation of the composition. The representations of the one or more non-target organisms may be predefined, received from a user, received from a datastore and/or another computer system, and/or otherwise received by system 1000.

In some implementations, system 1000 performs act 3015 by selector 200. In some implementations, system 1000 performs act 3015 at encoder 210, classifier 300, and/or via any other suitable module. The representations of the one or more pests and/or one or more non-target organisms may be used to condition the behavior of classifier 300. For example, system 1000 may select trained models 320a, . . . 320n of classifier 300 based on the representations of the one or more pests (e.g. by selecting such models based on which were trained over at least one of the one or more pests), as described in greater detail below. As another example, system 1000 may condition the behavior of classifier 300 by providing the representations of the one or more pests and/or the one or more non-target organisms as input to classifier 300, e.g. to inform predictions of synergistic efficacy of the candidate pesticidal composition against a pest and/or of toxicity between the candidate pesticidal composition and a non-target organism.

The candidate pesticidal compositions received, identified, generated, or otherwise obtained at acts 3005, 3010, and/or 3015 form an initial candidate pesticidal composition set (which may comprise representations received at 3005 and/or enhanced representations generated at 3010 and/or 3015). In some embodiments, system 1000 performs one or more filtration acts (such as optional filtration acts 3020, 3030, 3035, 3040 described herein) to determine a final candidate pesticidal composition set based on the initial candidate pesticidal composition set. Acts 3010 and/or 3015 may be performed before, after, and/or in parallel with one or more filtration acts; for example, system 1000 may enhance compounds' representations as described above after performing one or more filtration acts.

At 3020, system 1000 optionally filters the candidate pesticidal compositions (e.g. based on the representations received at 3005 and/or the enhanced representations generated at 3010) based on a compound exclusion criterion. For example, system 1000 may retrieve from a datastore (e.g. database 250 and/or 570) a list of compounds and/or atoms which are to be excluded from candidate pesticidal compositions. As one illustrative example, an example exclusion criterion may exclude compositions containing Arsenic and metals heavier than Calcium. As another illustrative example, applying an example exclusion criterion may comprise determining a measure of chemical complexity and excluding compositions containing a compound which for which that chemical complexity measure exceeds a threshold. For instance, such an exclusion criterion may exclude an alkane (or other organic acyclic) molecule with a chain length greater than a threshold. Such an exclusion criterion may comprise a rule (e.g. matching atoms with atomic masses greater than 40.078 or with an atomic number of 33), a list (e.g. a listing of Arsenic and all metals heavier than Calcium), a combination thereof, and/or any other suitable criterion. Exclusion criteria may be predefined and retrieved by system 1000 from a datastore (e.g. database 250, 570, and/or a parameter store (not shown)). In some embodiments, system 1000 retrieves a plurality of exclusion criteria at 3020. System 1000 may apply all of the retrieved exclusion criteria or select a subset to apply.

In some embodiments, system 1000 filters candidate pesticidal compositions based on a chemical complexity criterion at 3020. The chemical complexity criterion may comprise excluding compounds based on their chemical structure. For example, system 1000 may exclude compounds with a chemical structure comprising a number of atoms which is greater than a threshold (e.g. compounds with more than 50 atoms). The threshold may be predefined, provided by a user, generated by system 1000 (e.g. the threshold may be set to be equal to a measure of chemical complexity the 10^{th}, 20^{th}, 30^{th}, 40^{th}, 50^{th}, or another percentile of candidate compounds, ranked by a measure of complexity such as number of atoms), and/or otherwise obtained by system 1000. In some embodiments, system 1000 filters candidate pesticidal compositions based on a subset of such compositions' constituent compounds. For example, system 1000 may filter candidate pesticidal compositions based on a chemical complexity criterion applied against a candidate synergistic compound, without necessarily filtering such candidate pesticidal compositions based on a chemical complexity criterion applied against a candidate pesticidal compound.

In some embodiments, system 1000 filters candidate pesticidal compositions based on an ingredient whitelist criterion at 3020. For example, system 1000 may exclude any candidate pesticidal compositions comprising a compound having atoms not on a predefined list of non-excluded atoms. For example, system 1000 may be configured to increase the probability that selected candidate synergistic compounds are inert, and may exclude candidate pesticidal compositions where the candidate synergistic compound comprises an atom not on a list of atoms with high incidences in inert compounds. Such a list may comprise, for example, C, O, H, N, P, Cl, and F, as compounds with atoms outside that list tend to be more likely to have undesirable and/or unpredictable bioreactivity. In some embodiments, system 1000 filters candidate pesticidal compositions based on an ingredient blacklist criterion at 3020. For example, system 1000 may exclude any candidate pesticidal compositions comprising compounds having atoms on a predefined list of excluded atoms (e.g. such a list could comprise As, Sc, Ti, V, Cr, and atoms, e.g. heavy metals).

In some embodiments, system 1000 filters candidate pesticidal compositions based on a chemical property criterion at 3020. For example, system 1000 may exclude candidate pesticidal compositions comprising compounds with certain chemical properties, such as those which system 1000 identifies as highly flammable, unstable, and/or having certain known interactions with other compounds in the same candidate pesticidal composition (e.g. mixtures of atomic potassium and water). System 1000 may determine compounds' chemical properties based on, for example, an enhanced representation of the chemical compounds generated at acts 3010 and/or 3015, which may comprise records of such properties. System 1000 may also, or alternatively, retrieve chemical property information from a datastore such as database 250 and/or 570. Chemical property information may be retrieved from material safety data sheets (MSDS) for compounds of each candidate pesticidal composition.

In some embodiments, chemical property information is retrieved for each compound of a candidate pesticidal composition. In some embodiments, such information is retrieved for a subset of compounds of a candidate pesticidal composition. For example, in an implementation where system 1000 is configured to increase the probability that selected candidate synergistic compounds are inert, system 1000 may retrieve such information for candidate pesticidal compounds without necessarily retrieving such information for candidate synergistic compounds (e.g. where there is otherwise high confidence that candidate synergistic compounds are inert). As another example, in an implementation where system 1000 is configured to increase the probability that selected candidate synergistic compounds are inert, system 1000 may retrieve such information for candidate synergistic compounds in order to filter out candidate synergistic compounds with chemical properties which are likely to cause candidate synergistic compounds to be non-inert (e.g. where there is not otherwise high confidence that candidate synergistic compounds are inert), without necessarily retrieving such information for other compounds of the candidate pesticidal composition (e.g. where candidate pesticidal compounds are pre-selected and/or otherwise not individually subject to filtration.)

As noted above, such exclusions may be limited to a subset of compounds, such as by excluding candidate pesticidal compositions based on the atomic constituents and/or other chemical properties of candidate synergistic compounds and not necessarily other compounds. For example, suppose that compounds comprising heavy metal atoms are excluded; a composition with a candidate synergistic compound comprising a heavy metal might therefore be excluded, but a composition where the candidate synergistic compound lacks any heavy metal atoms might be accepted even if the composition also comprises a candidate pesticidal compound which comprises a heavy metal atom.

At 3030, system 1000 optionally determines the availability of one or more compounds from one or more datastores (e.g. database 570). Such datastores may comprise inventory systems, such as those provided by a user and/or by commercial chemical suppliers such as Sigma-Aldrich. System 1000 may query such datastores for availability of the one or more compounds. If a compound is identified as not available, and/or if its availability is less than an availability threshold, system 1000 may exclude candidate pesticidal compositions comprising that compound. Availability thresholds may be the same or different for different compounds and may be predetermined and/or provided by a user.

In some embodiments, at 3030 system 1000 additionally or alternatively retrieves a resource metric describing a per-unit resource allocation associated with one or more compounds. For example, system 1000 may retrieve a resource metric comprising a quantity of time required to synthesize, ship, and/or otherwise procure a quantity of a compound, a measure of synthesis complexity (e.g. number of atoms in the compound, which tends to correspond generally to the resources required to synthesize it), a quantity of funds required to procure the compound and/or its constituents, and/or any other suitable resource metric. System 1000 may exclude candidate pesticidal compositions which comprise a compound with an associated resource metric which exceeds a resource threshold. The resource threshold may, for example, be predetermined, provided by a user, and/or retrieved from another computer system. In some implementations, system 1000 generates an estimated composition resource metric based on one or more resource metrics associated with compounds of the candidate pesticidal composition and excludes candidate pesticidal compositions for which the associated estimated composition resource metric exceeds a resource threshold (which may be the same as or different than the resource threshold applied on a per-compound basis). System 1000 may generate the estimated composition resource metric for a candidate pesticidal composition based on, for example, determining a sum and/or a maximal value of the resource metrics of the compounds of the candidate pesticidal composition. System 1000 may scale, add to, or otherwise increase the estimated resource metric, e.g. based on a predetermined and/or user-supplied estimate of process overhead in preparing the candidate pesticidal composition from its constituent ingredients. In some implementations, system 1000 records candidate pesticidal compositions excluded due to exceeding a resource threshold and/or non-availability to a datastore (e.g. database 250 and/or 570). System 1000 may, for example, display such candidate pesticidal compositions to a user and/or generate a list of proposed future tests (e.g. ranked by resource metrics and/or availability).

At 3035, system 1000 optionally filters candidate pesticidal compositions based on a measure of each candidate pesticidal composition's similarity (or dis-similarity) to other candidate pesticidal compositions, e.g. to limit the selected candidate pesticidal compositions generated by method 3000 to those with similar candidate synergistic compounds. In one embodiment, the filtering may be performed using fingerprints for each compound, e.g. as described elsewhere herein. System 1000 may encode each candidate compound based on its fingerprint(s) (e.g. Morgan and/or MACCS fingerprints). For example, system 1000 may encode each candidate compound's molecular structure in bitmap form based on its fingerprints; system 1000 may determine a similarity measure between different compounds within the composition and/or between a compound in the composition and another compound (e.g. a compound previously excluded or included by system 1000) by determining a similarity measure between the bitmaps for the compared compounds. The similarity measure may be determined via any suitable similarity technique, such as by determining the Jaccard index between bitmaps (and/or between any other suitable representation of the compounds).

There are several modes of operation of system 1000 in performing act 3035. In some embodiments, system 1000 excludes compositions comprising compounds which have a similarity measure against any of one or more compounds which is greater than (or, in some embodiments, less than) a threshold. In some embodiments, system 1000 excludes composition comprising compounds which have a similarity measure against each of one or more compounds which is greater than (or, in some embodiments, less than) a threshold. In some embodiments, system 1000 includes only those compositions comprising compounds which have a similarity measure against any of one or more compounds which is greater than (or, in some embodiments, less than) a threshold. In some embodiments, system 1000 includes only those composition comprising compounds which have a similarity measure against each of one or more compounds which is greater than (or, in some embodiments, less than) a threshold. The threshold may, for example, be predetermined, provided by a user, and/or retrieved from another computer system. The mode of operation may be predetermined and/or selected by a user. For example, a threshold of 60% may be stored in a parameter store, optionally along with an “exclude <=threshold” option. In such a scenario, act 3035 could comprise excluding all candidate pesticidal compositions comprising compounds that did not meet at least 60% similarity test using the Jaccard index. A user may, by the application of appropriate settings, cause system 1000 to include or exclude similar or dissimilar compounds and candidate pesticidal compositions.

In some embodiments, system 1000 excludes candidate pesticidal compositions based on a similarity measure for a subset of the compounds of each candidate pesticidal composition. For example, system 1000 may exclude candidate pesticidal compositions based on a similarity measure of the candidate synergistic compound relative to a reference synergistic compound, without necessarily determining a similarity measure for other compounds of the candidate pesticidal composition. The reference synergistic compound may be provided by a user, predetermined, retrieved from another computer system, and/or otherwise obtained (e.g. the first candidate synergistic compound received by system 1000 while processing a batch of candidate pesticidal composition may be used as the reference synergistic compound). Restricting candidate synergistic compounds to those which are similar to a particular synergistic compound in this way may, in suitable circumstances, limit the number of unstable or otherwise impractical compounds selected by system 1000, as compounds with chemical similarity to a known stable compound (e.g. formic acid) tend to be more likely to also be stable, relative to arbitrary compounds.

In some embodiments, system 1000 determines a plurality of similarity measures and includes and/or excludes candidate pesticidal compositions based on the plurality of similarity measures. For example, system 1000 may determine a first similarity measure for a candidate synergistic compound (e.g. relative to a reference synergistic compound) based on a first fingerprint, such as a MACCS fingerprint. System 1000 may further determine a second similarity measure for the candidate synergistic compound (e.g. relative to the reference synergistic compound) based on a second fingerprint, such as a Morgan fingerprint. System 1000 may, for example, include the candidate pesticidal composition if both similarity measures are above a threshold (e.g. 50%, 60%, 70%, 80%, 90%, and/or some other suitable threshold, which may be the same or different for the two fingerprints) and exclude it otherwise.

At 3040, system 1000 optionally filters candidate compounds based on a toxicity criterion and/or suitability criterion. System 1000 may, for example, obtain for each compound of a candidate pesticidal composition a representation of toxicity, e.g. by retrieving it from the compound's received representation, enhanced representation, and/or from a datastore such as database 250 and/or 570. System 1000 may then exclude the candidate pesticidal composition if a compound of the candidate pesticidal composition has a corresponding representation of toxicity which satisfies the toxicity criterion. For example, system 1000 may exclude all candidate pesticidal compositions comprising compounds with any known toxicity. As another example, system 1000 may exclude candidate pesticidal compositions comprising compounds having certain types of toxicity (e.g. one or more toxicities identified by a dataset such as Tox21). As another example, system 1000 may exclude candidate pesticidal compositions comprising compounds having at least a threshold degree of toxicity (e.g. for a type of toxicity measured by a 5-point scale, system 1000 may exclude candidate pesticidal compositions comprising compounds having that type of toxicity with degree 2 or greater, without necessarily excluding those with degree 1). As another example, system 1000 may exclude candidate pesticidal compositions comprising compounds having toxicity against organisms on a list; for instance, if toxicity against humans and certain crops is considered undesirable, the list may comprise humans and those crops, but may exclude other organisms (e.g. pests, against which toxicity may be desired).

In some embodiments, act 3040 optionally comprises filtering candidate pesticidal compositions based on a suitability criterion. For example, system 1000 may retrieve from a datastore (such as database 250 and/or 570) a list of known-suitable and/or known-unsuitable compounds. System 1000 may exclude candidate pesticidal compositions comprising compounds which are listed as known-unsuitable, and/or may exclude candidate pesticidal compositions comprising compounds which are not listed as known-suitable. For instance, system 1000 may query an EPA-provided database of compounds that have been previously registered as pesticides and collect information about the previous registration such as the pests against which they are known to be effective. System 1000 may exclude any candidate pesticidal compositions which do not comprise at least one compound registered as effective against the one or more pests identified at 3015, and/or which are not registered as effective as a certain class of pesticide (e.g. in a fungicidal context, only compositions containing compounds known to be effective as fungicides generally may be included).

At 3045, system 1000 optionally selects one or more features of the candidate pesticidal composition and generates a reduced representation of the candidate pesticidal composition. For example, system 1000 may generate an enhanced representation at 3010 comprising a plurality of features, such as chemical properties (e.g. as generated via a QSAR model), and may select certain features for generation (in which case 3045 may be a constituent act of 3010) and/or remove one or more such features after generation (in which case 3045 may be a constituent or independent act and may occur at any suitable time).

Features which have been identified from among the thousands available as contributing to the accuracy of at least some embodiments of system 1000 in identifying synergistically efficacious pesticidal compositions include features relating to aromaticity, electronegativity, polarity, hydrophilicity/hydrophobicity, and hybridizations. In some embodiments, features are selected from one or more groups consisting of: electrostatic chemical features (and particularly: electronegativity of each atom of a compound, partial charges of a compound, valance molecular connectivity index (e.g. Chi index), aromaticity, and local dipole moments), topological chemical features (and particularly: hybridizations of atoms, graph distance index (e.g. Weiner index), and polarity number of bonds), conformational chemical features (and particularly: number of single bonds, number of double bonds, number of triple bonds, number of aromatic bonds, number of aromatic rings, orientation of functional groups, representations of cis-trans isomers, and representations of enantiomers), and surface-related and physiochemical properties (and particularly: a measure of a partition coefficient (e.g. log P), a measure of a distribution coefficient (e.g. log D), a measure of polar surface area, a measure of molecular surface area, an unsaturation index, a hydrophilic index, and a total hydrophobic surface area).

For instance, in at least one example embodiment, a large number of features (e.g. approximately 2000 in the case of the RDKit QSAR model) may be generatable for each of one or more constituent compounds of a candidate pesticidal composition via a QSAR model. Such features may include, for example, scalar properties (e.g. magnetic properties), two-dimensional matrix properties (e.g. functional groups), and/or three-dimensional matrix properties (e.g. geometric/conformational properties) for the compound.

System 1000 may select features which are expected to contribute to predictions of classifier 300. For example, system 1000 may select features which are correlated with pesticidal efficiency, and/or may remove features with low (or no) correlation with pesticidal efficiency. For instance, system 1000 may remove from the enhanced representation (e.g. by instructing the QSAR model not to generate) and/or cause the QSAR model not to generate features such as: a count of the number of iodine atoms in the compound, the molecular weight of the compound, and/or a count of the number of atoms in the compound.

As another example, system 1000 may select chemical features with variance exceeding a threshold, and/or may remove features with variance below the threshold. (E.g. in at least some embodiments, features which are identical across all compounds screened by system 1000 may be omitted, as they will have 0 variance.) In some embodiments, one or more categorical features are binarized; for example, a feature which describes the number of rings possessed by a compound which is dominated by the quantities 0 and 1 may be binarized to a feature describing whether or not the compound has rings (i.e. transforming the feature so that 0 maps to FALSE/0 and all other values map to TRUE/1). At 3050, system 1000 generates a final candidate pesticidal composition set based on representations of the candidate pesticidal compositions obtained at 3005, 3010, and/or 3015 and optionally based on the candidate pesticidal compositions excluded at 3020, 3030, 3035, and/or 3040. In some implementations, system 1000 performs the acts of method 3000 asynchronously. System 1000 may, in asynchronous and/or other embodiments, query a datastore, such as database 250, for records of candidate pesticidal compositions and/or constituent compounds and determines whether the records are ready for encoding by encoder 210. System 1000 may perform such queries periodically. System 1000 may determine that a record if ready for encoding when each of the other acts of method 3000 (excluding optional acts not provided by an embodiment) has been performed on the record's corresponding candidate pesticidal composition. In some embodiments, system 1000 excludes from the final candidate pesticidal composition set any candidate pesticidal compositions which have previously been encoded by encoder 210 and/or for which a prediction has been generated by classifier 300. System 1000 may optionally mark the records of such candidate pesticidal compositions to reflect such previous encoding and/or prediction and may, at 3050, retrieve that marking and exclude candidate pesticidal compositions accordingly.

In some embodiments, system 1000 filters any candidate pesticidal compositions that were used as part of a training set for trained models of classifier 300. System 1000 may store a list of previously-trained compounds and/or candidate pesticidal compositions in a datastore such as database 250.

After act 3050, method 3000 completes.

System 1000 may record representations of candidate pesticidal compositions received and/or generated at acts 3005, 3010, 3015, and/or 3050 to a datastore, such as database 250 and/or 570. The datastore may be available to other modules of system 1000, to a user, and/or to other computer systems. Where this disclosure recites other modules of system 1000 receiving information which is also recited herein as being stored to such datastores, receiving such information may comprise retrieving it from such datastores.

System 1000 may additionally or alternatively record candidate pesticidal compositions excluded at one or more filtering acts 3020, 3030, 3035, 3040 in a datastore, such as database 250 and/or 570. System 1000 may identify in such records that the candidate pesticidal composition and/or specific constituent compounds were excluded. System 1000 may record the reason for exclusion explicitly (e.g. by recording an indication that a compound was unavailable, on an exclusion list, or some other applicable reason) and/or implicitly (e.g. by recording compositions and/or compounds to different datastores depending on the reason for exclusion, so that compounds rejected for unavailability are recorded to one datastore, compounds rejected due to an exclusion list are recorded to another datastore, and so on). In some embodiments, system 1000 queries such datastore(s) and excludes candidate pesticidal compositions which were previously excluded prior to, in parallel with, and/or after applying filtering acts 3020, 3030, 3035, 3040.

### Encoding Candidate Pesticidal Compositions

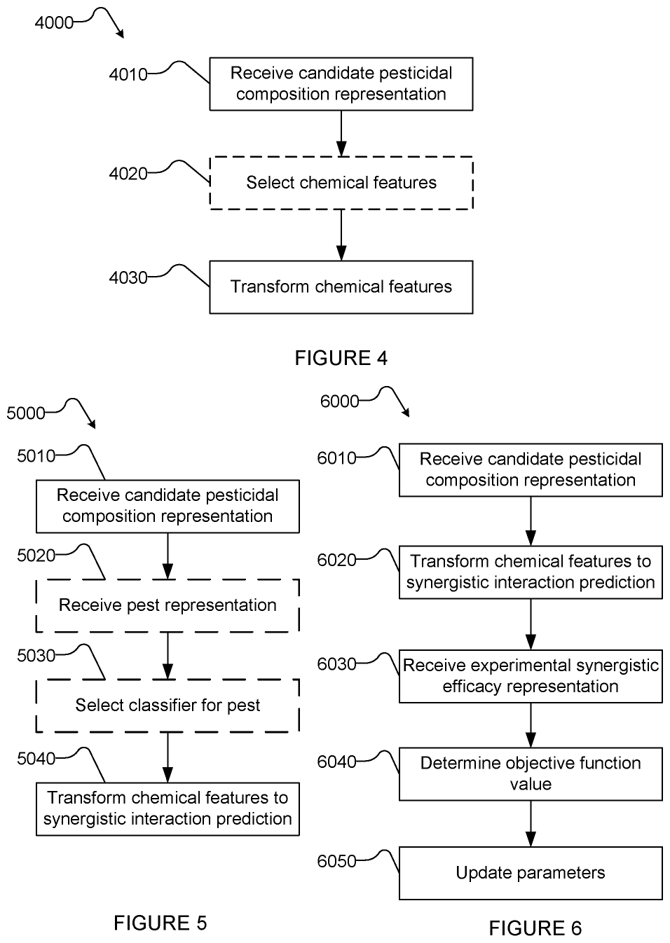

System 1000 encodes representations of candidate pesticidal compositions at encoder 210. FIG. 4 shows an example method 4000 for encoding representations of candidate pesticidal compositions which may be executed by encoder 210 and/or any suitably-configured computer system. At 4010, encoder 210 receives a representation of each candidate pesticidal composition, which may comprise received and/or enhanced representations of the candidate pesticidal composition's compounds, candidate pesticidal composition formulation parameters, fingerprints of compounds, graph representations of compounds, atomic information, molecular information (e.g. atom counts, bond type and bond counts), quantum mechanical information (e.g. electron charge distributions), and/or the other information about the candidate pesticidal composition and/or its constituent compounds as described herein. In at least some embodiments, encoder 210 receives a representation for each candidate pesticidal composition in the final candidate pesticidal composition set generated at act 3050 of method 3000. The representations of candidate pesticidal compositions received by encoder 210 are referred to for the purposes of describing encoder 210 as raw representations.

At 4030, system 1000 (e.g. at encoder 210) transforms each raw representation into an encoded representation of the candidate pesticidal composition. The encoded representation of the candidate pesticidal composition may comprise a unitary representation (e.g. a single latent vector) or a plurality of representations (e.g. one for each compound of the candidate pesticidal composition). The transformation effected by encoder 210 may comprise one or more of: compression, feature selection, and/or transcoding to generate encoded representations of candidate pesticidal compositions which are amenable to classification by classifier 300. For example, encoder 210 may transform atomic, molecular, quantum dynamical, and/or other information about candidate pesticidal compositions (including, e.g., features of constituent compounds) into a regularly-structured encoded representation which encodes at least a portion of that information while conforming to the structure required for input to classifier 300. For instance, the structure of the encoded representation may correspond to the structure of the input layer of a classifier 300 comprising a neural network (e.g. if classifier 300 takes 32-variable inputs with numerical values, the encoder may generate 32-variable encoded representations comprising numerical values, two 16-variable encoded representations comprising numerical values, and/or another set of encoded representations which align with the input required by classifier 300). The encoded representation is optionally lower-dimensional than the raw representation, and/or comprises fewer features than are provided by the raw representation, as described in greater detail below.

In some embodiments, encoder 210 compresses the raw representations of candidate pesticidal compositions. Raw representations of pesticidal compositions, including of their constituent compounds, tend to be complex and high-dimensional, comprising many datapoints. For example, enhanced representations of compounds which include QSAR-generated molecular information may provide in excess of 3000 variables—an intractably large number of variables for at least some computer systems to train over. Encoder 210 may transform such representations to lower-dimensional encoded representations of candidate pesticidal compositions.

For example, at least one illustrative embodiment of encoder 210 transforms raw representations having more than 3000 variables into encoded representations having 32 variables. Encoder 210 may be configured to transform raw representations into encoded representations having any number of variables (e.g. 10, 16, 20, 25, 30, 40, 50, 64, 100, 128, etc.). Such encoding may be lossless and/or lossy. A suitable encoder, such as those described below, may provide high degrees of reconstruction fidelity (i.e. low reconstruction loss), implying that in at least some embodiments the lower-dimensional representation can encode all or nearly all the information stored in the raw representation, albeit in encoded form.

Several types of encoders may be used without departing from the scope of the invention. For example, in at least some embodiments, encoder 210 compresses raw representations according to a compression technique such as Lempel-Ziv compression, prediction by partial matching, Huffman compression, arithmetic coding, Shannon-Fano compression, and/or the like.

Optionally, at 4020 system 1000 (e.g. at encoder 210) performs feature selection based on the raw representations. Such feature selection may be in addition to, or instead of, the feature selection of act 3045 of method 3000. (Act 3045 may, optionally, be performed in whole or in part by encoder 210.) Encoder 210 may, for example, discard portions of a raw representation and retain other portions of the raw representation to produce a lower-dimensional encoded representation comprising only the retained portion. Although feature selection is a form of (usually lossy) compression, the retained portion is not necessarily compressed or otherwise encoded (although encoder 210 may optionally encode the retained portion, e.g. as described herein).

In some implementations, feature selection by encoder 210 comprises extracting, based on the raw representation, one or more feature descriptors. A feature descriptor describes a feature of the candidate pesticidal composition (e.g. a feature of a constituent compound of the candidate pesticidal composition) and may comprise, for example, atomic information, molecular information (e.g. atom counts, bond type and/or bond counts), quantum mechanical information (e.g. electron charge distributions), and/or other features of the candidate pesticidal composition (e.g. of its constituent compounds). A given feature descriptor may be associated with one or more candidate pesticidal compositions. A plurality of feature descriptors may be associated with each other, such as when a plurality of feature descriptors are associated with a fingerprint (e.g. a graph representation) of a compound of a candidate pesticidal composition.

In some implementations, encoder 210 generates encoded representations comprising explicit representations of feature descriptors. For example, encoder 210 may extract an atom count from a raw representation of a compound of candidate pesticidal composition and generate an encoded representation comprising a value explicitly representing that atom count. For instance, if the raw representation of a candidate pesticidal composition indicates that a first compound of the candidate pesticidal composition has 10 atoms, encoder 210 may generate an encoded representation which comprises the numerical scalar value 10. As another example, feature descriptors may comprise non-scalar (e.g. vector) values, such as where encoder 210 encodes a compound's molecular structure in the encoded representation as a simplified molecular-input line-entry system (SMILES) string. In some implementations, encoder 210 generates encoded representations comprising implicit representations of feature descriptors, e.g. via a compressed representation which may combine feature descriptors into one scalar value and/or distribute the information of a feature descriptor across a plurality of scalar values. The latent space encoded representation generated by the embodiment of encoder 210 comprising an encoder portion of a variational autoencoder is an example of such implicit feature selection.

The features selected by encoder 210 may vary by embodiment. For example, atomic, molecular, quantum dynamical, and/or other features of candidate pesticidal compositions (e.g. features of their constituent compounds) may be encoded differently by different encoders 210 and/or by a single encoder 210 providing different encoding schemes. Various encodings may be provided by encoders 210. System 1000 may generate an encoded representation for a compound using more than one encoding if desired, and/or may generate encoded representations for different compounds using different encoders 210 and/or different encodings provided by an encoder 210. In some implementations, system 1000 provides at least two encoders—at least a first encoder for transforming raw representations of pesticidal compounds and at least a second encoder for transforming raw representations of synergistic compounds. Such first and second encoders may provide different encodings (e.g. the pesticidal and synergistic compounds may be encoded with different numbers of values, with different selected features, based on different trained parameters for the encoders, and/or by different types of encoders).

In some embodiments, encoder 210 is configured to encode a candidate pesticidal composition comprising more than two constituent compounds (e.g. comprising multiple candidate pesticidal compounds, multiple candidate synergistic compounds, and/or one or more other compounds, such as adjuvants, solvents, etc.). For example, encoder 210 may generate an encoded representation based on three, four, or more compounds. In some embodiments, encoder 210 receives a fixed number of representations of compounds (e.g. encoder 210 may be configured to receive three compounds) and is trained over training data comprising representations of pesticidal compositions having the same number of compounds. In some embodiments, encoder 210 receives a variable number of compounds depending on the number of constituent compounds of a candidate pesticidal composition being encoded. Encoder 210 may encode such compositions in any appropriate way; for example, encoder 210 may receive a fixed number (e.g. one, two, or more) of representations of compounds at each pass of an encoding process to generate intermediate encoded representations (e.g. 16-, 32-, 64-, or 128-variable floating-point representations) and may then generate a final encoded representation (e.g. of the same form as the intermediate encoded representations) by combining the intermediate encoded representations via an attention mechanism, a pointwise sum, and/or any other suitable approach. Encoder 210 may optionally generate separate encoded representations for candidate synergistic compounds and for candidate pesticidal compounds.

In at least one example embodiment, encoder 210 receives a set of identifications of feature descriptors required by classifier 300 (which may comprise, e.g. in the case of classifier 300 comprising an ensemble classifier, identifications of feature descriptors required by trained classifiers 310a, . . . 310n) and performs feature extraction for each compound represented in the raw representation of a candidate pesticidal composition based on the set of identifications of feature descriptors. The set of identifications may comprise an identification of a number of compounds accepted by classifier 300 and/or, for each compound, a set of feature descriptors of the compound, and encoder 210 may perform feature extraction for each compound based on the set of feature descriptors specified for that compound. In some implementations, encoder 210 adds mixture ratio information associated with the candidate pesticidal composition (e.g. as represented by and/or associated with the raw representation) to the encoded representation. For example, encoder 210 may encode representations of compounds, add these to an encoded representation, and add mixture ratio information to the encoded representation of the candidate pesticidal composition independently of the compounds' encodings. As another example, mixture ratio information may be encoded together with compounds' representations, e.g. by incorporating such mixture ratio information into a compressed and/or latent space representation (described below) generated by encoder 210. For instance, encoded representations of the compounds may be combined (optionally along with mixture ratio information) via concatenation, an attention mechanism, and/or any other suitable combination technique.

In some embodiments, some information passed to classifier 300 is not encoded. For example, encoder 210 may encode only the raw representations of candidate compounds, whereas other information (such as candidate pesticidal composition formulation parameters and/or representations of the one or more pests) may be passed to classifier 300 without encoding. In some embodiments, system 1000 encodes such other information separately from the encoding of compounds' raw representations.

In some embodiments, encoder 210 receives a raw representation of a compound as input and transforms the raw representation based on a set of trained parameters of encoder 210. In some embodiments, encoder 210 receives and encodes raw representations of each compound of a candidate pesticidal composition independently, thereby generating an encoded representation for each compound. In some embodiments, system 1000 provides a plurality of encoders 210. System 1000 may encode a first compound of a candidate pesticidal composition (e.g. a pesticidal active ingredient) with a first encoder and encode a second compound of the candidate pesticidal composition (e.g. a candidate synergistic ingredient) with a second encoder. The first and second encoders may be trained over the same or different training sets and comprise the same or different structure and/or parameters. For example, the first encoder may be trained over a training set of pesticidal active ingredients and the second encoder may be trained over a training set of synergistic (and/or antagonistic and/or non-synergistic) ingredients.

In some embodiments, encoder 210 comprises at least a portion of a variational auto-encoder. In at least one embodiment, encoder 210 comprises an encoder portion of a variational auto-encoder which has been trained together with a decoder portion but operates without the decoder portion during encoding. (The decoder portion does not necessarily form part of system 1000.) Such an encoder 210 transforms (relatively sparse) raw representations x in an input space X characterized by the input data to (relatively dense) encoded representations z in a latent space Z characterized by a prior distribution p(z). In particular, encoder 210 determines p(z|x) to generate a distribution over the latent space for a given compound. Encoder 210 may transform that distribution into an encoded representation in any suitable manner. In at least some implementations, encoder 210 transforms the distribution deterministically into the encoded representation, e.g. by determining a mean value for the distribution (e.g. either independently or jointly over the latent variables). Such an encoder 210 can be considered to provide an implicit feature compression by tending to identify those features which most contribute to accurate reconstruction (and are in some sense the compounds' “distinguishing” features).

In some embodiments, encoder 210 comprises an encoder of an inverse autoregressive flow variational autoencoder. For example, encoder 210 may be trained over any suitable training data set of chemical compositions (as described elsewhere herein) to find parameters which minimize a suitable objective function. For instance, the objective function may be provided by log p(x) (and, e.g., a loss function may be derived therefrom via negation), which in at least some embodiments may be approximated by a lower bound based on:

$$- {E}_{q} [ log \frac{q ( {z}_{T} \vert x )}{p ( {z}_{T} , x )} ]$$

which may be expressed in the form:

E_{q}[log p(x|z_{T})+log p(z_{T})−log q(z_{T}|x)]

where p is the true distribution which the inverse autoregressive flow variational autoencoder is trained against, q is the approximating distribution which the inverse autoregressive flow variational autoencoder learns, z_{T} is an element of the latent space and may be described in at least some embodiments as the T^{th} zᵢ where z₀˜q(z₀|x) and zᵢ=fᵢ(zᵢ₋₁, x) for some series of invertible transformations fᵢ(⋅), and x is an element from the input space.

Moreover, in at least some embodiments, log q (z_{T}|x) and log p(z_{T}) may be approximated as:

$$log q ( {z}_{T} \vert x ) = - {\sum }_{i = 0}^{D} [ \frac{1}{2} {ϵ}_{i}^{2} + \frac{1}{2} log 2 π + {\sum }_{t = 0}^{D} log {σ}_{t , i}^{- 1} ]$$

$$log p ( {z}_{T} ) = - {\sum }_{i = 0}^{D} [ \frac{1}{2} {z}_{T}^{2} + \frac{1}{2} log 2 π ]$$

where ϵ is a suitable noise vector (e.g. ϵ˜ [symbol] (0, l)) and σ_{t,i} is the variance for the i^{th} element of latent variable zₜ.

In some embodiments, encoder 210 is trained via a semisupervised approach, for example to minimize a reconstruction loss between input representations in the training set and reconstructed representations generated by the decoder portion (based on encoded representations generated by encoder 210). In some embodiments, encoder 210 is pre-trained and/or trained over a larger and/or more general dataset than classifier 300. For example, classifier 300 may be trained over pesticidal compositions (and/or over subclasses of such compositions), whereas encoder 210 may be trained over a chemical dataset which is not limited to, and may not even contain, pesticidal compositions. In some embodiments, encoder 210 and classifier 300 are trained together, such that training involves updating the parameters of both encoder 210 and classifier 300 to minimize (or maximize, as appropriate) a shared objective function over shared data. For example, training data may comprise a classifier-relevant subset, and a combined loss function for encoder 210 and classifier 300 may be based on: [symbol] _{combined}= [symbol] _{encoder}+α [symbol] _{classifier}, where α=1 if a given datum is in the classifier-relevant subset and α=0 otherwise. In some embodiments, encoder 210 and classifier 300 are trained separately. A potential advantage of training encoder 210 and classifier 300 together, relative to training them separately, is training together may tend to cause encoder 210 to tend to select features which are more relevant to classifier 300, at a potential cost of greater complexity and limited relevant training data.

In some embodiments, encoder 210 comprises a neural network, such as a graph convolutional neural network. The neural network may receive a raw representation for a compound as input (and/or a portion thereof, e.g. encoder 210 may receive a graph representation of the compound with associated properties) at an input layer and transform the raw representation based on a set of trained parameters corresponding to the input layer and based on the form of activation function and non-linearity provided by the neural network, thereby generating an intermediate representation. Encoder 210 may further transform the intermediate representation via one or more hidden layers, each with corresponding structure (e.g. inter-layer inputs/outputs), non-linearities, and trained parameters, and finally generate the encoded representation at an output layer (having its own structure, non-linearities, and trained parameters form). In at least some embodiments, the structure of the output layer corresponds to the form of input required by classifier 300. For example, if classifier 300 receives a 32-variable input, encoder 210 may generate a 32-variable encoded representation via a 32-variable output layer. (Intermediate representations do not necessarily, and usually will not, have the same number of variables or the same structure as the output layer).

In some embodiments, classifier 300 comprises encoder 210 (i.e. encoding and classifying functionality may be provided by one module). For example, in some embodiments classifier 300 may comprise a graph convolutional neural network (GCNN) which receives one or more graph representations for a candidate pesticidal composition (e.g. as produced by selector 200) and, at an initial stage, flattens those representations by traversing the graph(s), accumulating information at their nodes and/or edges, and thereby determining an intermediate (i.e. encoded) representation of the candidate pesticidal composition. At a later stage of the GCNN's operation, the intermediate representation is further transformed into an appropriate output.

For example, system 1000 may generate and provide to the GCNN a graph representation for each compound of the candidate pesticidal composition. As another example, system 1000 may generate and provide to the GCNN one graph representation for the candidate pesticidal composition, which may comprise disjoint subgraphs representing each compound of the candidate pesticidal composition. In some embodiments, system 1000 may connect such disjoint subgraphs, thereby generating a connected graph representing at least a portion of the candidate pesticidal composition. In at least one embodiment, system 1000 adds edges (representing bonds) between hydrogen bonding sites in the graph representations of the candidate pesticidal composition's constituent compounds. System 1000 may represent bond length in such graph representations; the representation of the added bonds between hydrogen bonding sites may be provided with different length than single and double bonds. For instance, bond length may be represented categorically, in which case the length single bonds could be 1, of double bonds could be 2, and of the added bonds could be 3 (or, in a one-hot encoding, as (1,0,0), (0,1,0), and (0,0,1), respectively). As another example, bond length may be represented continuously (e.g. based on physical length), in which case the length of the added bonds could be represented as longer (i.e. weaker) than a single bond (e.g. 1 for a single bond, 0.5 for a double bond, and 2 for an added bond). Representing bond length of added bonds distinctly from that of single bonds has, in at least some experimental tests, correlated with improved performance of herein-described systems and methods.

System 1000 may record encoded representations of candidate pesticidal compositions generated by encoder 210 to a datastore, such as database 250 and/or 570. Encoded representations may be associated with their corresponding raw representations (e.g. with the corresponding received representation and/or representation identified at act 3050 of method 3000). Encoded representations may also, or alternatively, be associated with the encoder (e.g. encoder 210) which generated the encoded representation. Such associations may comprise, for example, recording an identifier of the corresponding representation/encoder in the record of the encoded representation, and/or recording an identifier of the encoded representation in record(s) of the associated representation/encoder. The datastore may be available to other modules of system 1000 (e.g. classifier 300), to a user, and/or to other computer systems. Where this disclosure recites other modules of system 1000 receiving information which is also recited herein as being stored to such datastores, receiving such information may comprise retrieving it from such datastores. In some embodiments, if encoder 210 is modified (e.g. by updating its trained parameters through training), system 1000 may re-generate encoded representations associated with encoder 210 by obtaining from the datastore the raw representations (and/or, e.g., obtaining from selector 200 such raw representations based on received representations) and transforming the raw representations to new encoded representations. For example, if system 1000 provides multiple encoders, this may reduce the computational requirements for re-encoding relative to re-encoding all encoded representations for all encoders.

### Generating Synergy Predictions for Candidate Pesticidal Compositions

Classifier 300 receives, for each candidate pesticidal composition, the encoded representation generated by encoder 210 and generates one or more predictions based on the encoded representation and based on one or more sets of trained parameters. FIG. 5 shows an example method 5000 for generating a prediction of synergistic efficacy of a candidate pesticidal composition against one or more pests which may be executed by classifier 300 and/or any suitably-configured computer system. At 5010, classifier 300 receives a representation of each candidate pesticidal composition, which may comprise received, enhanced, and/or encoded representations of the candidate pesticidal composition (and may comprise such representations of the composition's constituent compounds). At 5040, classifier 300 transforms such representations to a prediction of synergistic interaction of the candidate pesticidal composition's constituent compounds against one or more pests. Classifier 300 models complex non-linear relationships between candidate compounds which form the basis of synergistic and/or antagonistic interactions between compounds of a candidate pesticidal composition upon one or more pests. For example, an active ingredient may be effective against a specific pest in the lab, but is unable to penetrate a cellular membrane of the pest in an in planta or in field context due to natural defenses of the pest. A synergistic combination of two or more compounds (e.g. one or more active compounds and one or more synergistic compounds) permits the active compound(s) access to the pest's cellular structure, thereby rendering the active compound effective for in planta and field usage. Such interactions between compounds and pests are not readily predicted, even by subject matter expects.

Classifier 300 may comprise any suitable classifier, such as a neural network, a decision tree, logistic regression, support vector machine, a stacking model classifier, and/or any other suitable classifier. In some embodiments, including the depicted embodiment of FIG. 1, classifier 300 comprises an ensemble classifier comprising a plurality of trained classifiers 310a . . . 310n (collectively and individually “classifiers 310”), each of which generates a prediction based on a corresponding set of trained parameters 320a . . . 320n (collectively and individually “trained parameters 320”). In some embodiments, classifiers 310 comprise deep neural network (DNN) models with a plurality of computational layers. Each classifier 310 models interactions between compounds and also interactions between one or more of the compounds and natural defenses of one or more pests. System 1000 may comprise any number of classifiers 310. For example, system 1000 may comprise 8, 16, 32, 64, 128, and/or any other suitable number of classifiers (which need not be a power of two).

For example, classifier 300 may comprise a plurality of trained neural network classifiers (e.g. classifiers 310), each of which is parametrized by a corresponding set of trained parameters 320 (e.g. classifier 310a may be parameterized by trained parameters 320a, classifier 310b may be parameterized by trained parameters 320b, and so on). Different classifiers 310 (and thus different trained parameters 320) may be trained over different pests and/or different compounds and may thereby model different interactions. For example, trained parameters 320 for each classifier 310 may have been trained on a corresponding training dataset comprising compositions of compounds (and, optionally, one or more pests) that have been identified as having synergistic and/or antagonistic effects. In some embodiments of method 5000, system 1000 receives one or more representations of the one or more pests (at 5020) and selects classifiers 310 trained over at least one of the one or more pests (at 5030), e.g. as described in greater detail elsewhere herein. The selected classifiers 310 are then executed to generate predictions at 5040.

FIG. 6 shows an example method 6000 for training parameters of classifier 300. Method 6000 may optionally comprise training parameters of encoder 210 (e.g. by training encoder 210 and classifier 300 together and/or by training encoder 210 substantially in accordance with the following description of method 6000.) In some embodiments act 6010 substantially corresponds to act 5010. In some embodiments method 6010 comprises selecting candidate pesticidal composition representations based on a synergistic interaction prediction (such as a synergistic interaction prediction generated as in act 5040 and/or act 6020.) For example, in some embodiments method 6000 comprises training parameters of classifier 300 via active learning, which may comprise, for example, determining an importance value for each of a plurality of candidate pesticidal composition representations (e.g. all available candidate pesticidal composition representations, candidate pesticidal composition representations within a batch, candidate pesticidal composition representations having corresponding synergistic interactions predictions with variance exceeding a threshold, or any other suitable plurality) based on synergistic interaction predictions generated (e.g. as in acts 5040 and/or 6020) for each such candidate pesticidal composition representations. In some embodiments, one or more of the candidate pesticidal composition representations are selected at act 6010 based on their corresponding importance values and acts 6020, 6030, 6040, and 6050 are performed on the basis of the selected candidate pesticidal composition representations, thereby updating the parameters of classifier 300 based on the selected candidate pesticidal composition representations.

In some embodiments, determining importance values for a plurality of candidate pesticidal composition representations comprises determining an informativeness metric for each candidate pesticidal composition representation of the plurality. The informativeness metric may be based on (and in some embodiments is identical to) a standard deviation, variance, and/or confidence interval of one or more synergistic interaction predictions generated by classifier 300 (e.g. as in acts 5040 and/or 6020) for the candidate pesticidal composition representation. In some embodiments, such as those where classifier 300 comprises an ensemble classifier, variance may be determined as described elsewhere herein with reference to a standard deviation 7220, variance, and/or confidence interval 7220) and/or by any other suitable determination. In at least one embodiment, the importance metric comprises determining a variance (e.g. based on standard deviation 7220). In some embodiments, such as those comprising a hyperplane-based classifier 300, the informativeness metric may be based on a distance of a candidate pesticidal composition representation to the nearest hyperplane. In some embodiments, other suitable measures of importance may be additionally or alternatively be determined.

In some embodiments selecting candidate pesticidal composition representations further comprises selecting candidate pesticidal composition representations based on a representativeness criterion. For example, candidate pesticidal composition representations may be clustered based on a similarity metric (e.g. graph similarity, for at least some embodiments where candidate pesticidal composition representations comprise a graph representation of candidate molecules and/or other composition substituents) and one or more candidate pesticidal composition representations may be selected from each of a plurality of the clusters. In some embodiments informativeness metrics are determined for only a subset of candidate pesticidal composition representations within a cluster; for example, informativeness metrics may be determined for the candidate pesticidal composition representation at the center (as defined by the clustering metric) of each cluster and candidate pesticidal composition representations from the plurality of clusters may be selected based on their informativeness metric (e.g. by selecting the n candidate pesticidal composition representations with the highest or lowest importance value, as appropriate; by selecting candidate pesticidal composition representations with an importance metric above or below (and/or, optionally, equal to) a threshold, as appropriate; and/or by any other suitable selection criterion).

A suitable representativeness criterion can promote dissimilarity between selected candidate pesticidal composition representations and can, in suitable circumstances and optionally in combination with a suitable informativeness metric, enable the training classifier 300 to reach model convergence with fewer labelled candidate pesticidal composition representations than would be required by random sampling. Obtaining labelled candidate pesticidal composition representations can be costly; for instance, it may involve human experts performing laboratory experiments to confirm synergistic interactions for candidate pesticidal compositions. Such an active learning approach can, in suitable circumstances, reduce the quantity of laboratory experimentation necessary or desirable to train the model adequately.

In some embodiments act 6020 substantially corresponds to act 5040. In some embodiments classifier 300 operates in a different mode at act 6020 than at act 5040, such as in embodiments where classifier 300 generates predictions with dropout during training at act 6020 but not at act 5040.

At 6030, system 1000 receives a representation of an experimental result comprising an indication of synergistic and/or antagonistic efficacy of the candidate pesticidal compositions of act 6010 against at least one training pest. In some embodiments, the at least one training pest is one of the one or more pests for which classifier 300 generates predictions. In some embodiments, the at least one training pest shares a pesticidal mode of action with at least one of the one or more pests. For example, if the one or more pests for which classifier 300 generates predictions include lepidopteran pests (such as the codling moth), classifier 300 may be trained over experimental results comprising indications of synergistic and/or antagonistic efficacy of candidate pesticidal compositions against other pests sharing pesticidal modes of action with such lepidopteran pests, such as related lepidopteran pests (e.g. in the earlier example involving codling moth, such related lepidopterans could include pink bollworm).

At 6040, system 1000 determines a value for an objective function (which may comprise, for example, a loss function) based on the prediction generated at 6020 and the representation of an experimental result received at 6030, e.g. based on a difference between them. At 6050, system 1000 updates the parameters of classifier 300 based on value of the objective function value determined at 6040, e.g. via backpropagation. In some implementations, different classifiers 310 have been trained over different subsets of a common training dataset. The subsets may be overlapping or disjoint. (Each classifier may further be validated against the elements of the common training set over which it was not trained.) Subsets may be determined pseudorandomly, by identifying subranges based on some ordering of the dataset, and/or by any other suitable determination criteria.

In some implementations, subsets of the common training dataset may have been determined based on pests for which compositions have been tested for synergistic (and/or antagonistic) interaction. For example, a first classifier 310a may have been trained over a first subset of training data comprising compositions with known synergistic, antagonistic, or no interaction for at least a first pest. A second classifier 310b may have been trained over a second subset of training data comprising compositions with known synergistic, antagonistic, or no interaction for at least a second pest. Classifiers 310a and 310b may have been trained against interactions for the first and second pests, respectively. For instance, classifier 310a may have been trained to generate predictions of synergistic effect of a composition for at least the first pest which minimize a reconstruction loss (or other suitable objective function) over the first subset of training data, whereas classifier 310b may have been trained to generate predictions of synergistic effect of a composition for at least the second pest which minimize a reconstruction loss (or other suitable objective function) over a second subset of training data. Classifier 310a is referred to herein as being trained against the first pest, and classifier 310b as being trained against the second pest. In some implementations, classifiers 310 are trained against classes of pests—for example, first classifier 310a may have been trained against fungal pests and classifier 310b may have been trained against bacterial pests.

Alternatively, or in addition, subsets of the common training dataset may have been determined based on the chemical properties of compositions in the common training dataset, such as the chemical structure of constituent compounds. Mixtures may be grouped into subsets based on, for example, their broad chemical class (e.g. organic, inorganic, synthetic, and/or biological), particular chemical functional group (e.g. possessing an aryl, alkyl, ethyl, methyl, and/or other group), similarity (e.g. a representative compound and its substituents, isomers, other compounds with which it shares a moiety, and other structurally related compounds), physical state of the compositions and/or its constituent compounds (e.g. fumigants, spray, dust, etc.). For example, a first classifier 310a may have been trained over a first subset of training data comprising compositions comprising an organic pesticidal active ingredient. A second classifier 310b may have been trained over a second subset of training data comprising compositions comprising an inorganic pesticidal active ingredient. Classifiers 310a and 310b may have been trained against organic and inorganic pesticidal active ingredients respectively. For instance, classifier 310a may have been trained to generate predictions of synergistic effect of compositions comprising organic pesticidal active ingredients (e.g. against one or more pests) which minimize a reconstruction loss (or other suitable objective function) over the first subset of training data, whereas classifier 310b may have been trained to generate predictions of synergistic effect of compositions comprising inorganic pesticidal active ingredients (e.g. against the same or different pest(s) as the first classifier) which minimize a reconstruction loss (or other suitable objective function) over the second subset of training data. In some implementations, classifiers 310 are trained against classes of pests—for example, first classifier 310a may have been trained against fungal pests and classifier 310b may have been trained against bacterial pests.

System 1000 may store, receive during operation, and/or be operable to retrieve a record indicating which compound(s) and/or pest(s) each classifier 310 has been trained against. In some embodiments, classifier 300 selects one or more classifiers 310 from a plurality of classifiers 310 based on the candidate pesticidal composition to be processed (e.g. based on the received, enhanced, raw, and/or encoded representation of the candidate pesticidal composition) and generates predictions with the selected classifiers 310 based on their associated parameters 320 and based on the encoded representation of the candidate pesticidal composition. For example, if classifier 300 is predicting a likelihood of synergistic effect of a candidate pesticidal composition against varroa mites, and if classifiers 310a and 310b have been trained against varroa mites and classifier 310c has not, then classifier 300 may select and generate predictions with classifiers 310a and 310b (based on parameters 320a and 320b) without necessarily selecting or generating predictions with classifier 310c. As another example, if a candidate pesticidal composition comprises an active ingredient against which classifiers 310b and 310c have been trained (e.g. compositions comprising that compound and various synergistic compounds) and classifier 310a has not, then classifier 300 may select and generate predictions with classifiers 310b and 310c (based on parameters 320b and 320c) without necessarily selecting or generating predictions with classifier 310a.

In some embodiments, classifier 300 selects and retrieves trained parameters 320 from a trained parameter database 251. Each classifier 310 independently generates a prediction of synergistic (and/or antagonistic) interaction based on the corresponding trained parameters 320. The prediction may comprise, for example, a probability (and/or confidence interval) of such synergistic interaction, a degree of such synergistic interaction, and/or a metric value (e.g. a MIC and/or FICI value) describing such synergistic interaction. Classifiers 310 are not limited to generating a prediction and may generate additional and/or alternative output; for example, classifiers 310 may also (or alternatively) predict toxicity and/or volatility of the candidate pesticidal composition (and/or of any constituent compounds), resistivity of a pest to the candidate pesticidal composition (e.g. based on pest genomic data received as input and/or by training classifiers 310 against pests' resistivity). The prediction (and/or other output) from each classifier 310 may be sent to combiner 400 for combination.

In some embodiments, classifier 300 (e.g. at least one classifier 310) is stochastic and can produce different predictions run-to-run based on one encoded representation. In some implementations, classifier 300 generates more than one prediction (e.g. by a given classifier 310, in the case of an ensemble classifier) based on one encoded representation. For example, system 1000 may perform dropout during inference with classifier 300, e.g. by pseudorandomly deactivating variables of the model(s) of classifier 300 (e.g. of at least one classifier 310) during inference. (Dropout may optionally also be performed in training.) Each iteration of inference may thus be expected to produce different results. System 1000 may combine a plurality of such predictions to determine a combined prediction and may assign to the combined prediction a confidence based on a variance of the plurality of predictions, e.g. as described in greater detail elsewhere herein.

In some embodiments, classifier 300 receives an encoded representation (e.g. from encoder 210), optionally determines a number N of classifiers 310 to select, optionally determines a number M of predictions to generate for each classifier 310 (N and M as described below), selects N classifiers 310 if appropriate (e.g. based on the encoded representation, and/or as described above), and generates M predictions with each of the N selected classifiers 310 based on the encoded representation and trained parameters 320 corresponding to selected classifiers 310. The number N of classifiers 310 to select and/or the number M of predictions to generate for each classifier 310 may be predetermined, provided by a user, determined by system 1000 (e.g. based on available computing resources), and/or otherwise obtained by classifier 300. For example, N may be 8, 16, 32, 64, 128, and/or any other suitable number (not necessarily a power of two). M may be 20, 40, 100, 200, 1000, and/or any other suitable number (not necessarily a multiple of 10). In at least one embodiment, N is 32 and M is 100. The terms N and M may be implicit in the model; for example, classifier 300 may be configured to generate one prediction with each classifier 310 (i.e. N=n and M=1). Classifier 300 may select the N trained classifiers 310 (and the corresponding trained parameters 320 from trained parameter database 251) based on the encoded representation, e.g. as described above. Classifier 300 parameterizes classifiers 310 using the selected trained parameters 320 and generates predictions based on the selected trained parameters 320.

System 1000 may record predictions generated by classifiers 310 to a datastore, such as database 250 and/or 570. Predictions may be associated with their corresponding encoded representations (e.g. with the corresponding received representation, raw representation, and/or encoded representation). Predictions may also, or alternatively, be associated with the classifier 300 (and/or classifier 310) which generated the prediction. Such associations may comprise, for example, recording an identifier of the corresponding representation/classifier in the record of the prediction, and/or recording an identifier of the prediction in record(s) of the associated representations/classifier 300/310. The datastore may be available to other modules of system 1000 (e.g. combiner 400), to a user, and/or to other computer systems. Where this disclosure recites other modules of system 1000 receiving information which is also recited herein as being stored to such datastores, receiving such information may comprise retrieving it from such datastores. In some embodiments, if the corresponding encoded representation and/or classifier 300 (and/or classifier 310) for a prediction is modified (e.g. by updating trained parameters 320 through training), system 1000 may re-generate the prediction by obtaining from the datastore the corresponding encoded representation (and/or, e.g., obtaining from another module such encoded representations, including by re-generating them at such other module) and transforming the encoded representations to new predictions via classifier 310. This may reduce the computational requirements for regenerating predictions relative to regenerating all predictions for all classifiers 310 and/or all encoded representations.

### Combining Synergy Predictions

In at least some embodiments, combiner 400 combines a plurality of predictions generated by classifier 300 into a final prediction 450. In some implementations, prediction 450 comprises a measure of probability of a synergic and/or antagonistic interaction between compounds of a candidate pesticidal composition and/or one or more pests. For example, prediction 450 may comprise a mean and confidence interval. In at least some implementations wherein classifier 300 comprises a plurality of classifiers 310, combiner 400 generates prediction 450 based on the predictions of each classifier 310.

An exemplary data flow characterizing a method of operation of combiner 400 is illustrated in FIG. 7. Combiner 400 receives a plurality of predictions 7100 and generates a combined prediction 7300 based on predictions 7100. In at least the depicted embodiment, combiner 400 receives a plurality of predictions 7100 comprising a plurality of predictions 7110 generated by each classifier 310 of classifier 300 (these are depicted as rows of predictions 7110 in a matrix of predictions 7100 in the depicted data flow of FIG. 7). In some implementations, each classifier 310 may generate a number M of predictions 7110 over the course of M iterations. Predictions 7100 may thus comprise a plurality of predictions 7120 generated for each iteration (these are depicted as columns of predictions 7120 in matrix of predictions 7100 in the depicted data flow of FIG. 7). The number of predictions 7120 for each iteration may be the same, e.g. N, for each iteration, or may differ between iterations, e.g. in embodiments where a classifier 310a generates predictions over more or fewer iterations than another classifier 310b.

In some embodiments, combiner 400 generates a plurality of aggregate predictions 7200 based on predictions 7100 and generates combined prediction 7300 based on aggregate predictions 7200. Combiner 400 may generate aggregate predictions 7200 by, for example, identifying a plurality of subsets of predictions 7100 and generating for each such subset an aggregate prediction based on the predictions 7100 of that subset. For example, combiner 400 may identify each plurality of predictions 7110 generated by a classifier 310 and/or each plurality of predictions 7120 associated with an iteration as subsets and may generate each of the aggregate predictions 7200 based on a corresponding plurality of predictions 7110 and/or 7120. Combiner 400 generating an aggregate prediction 7200 may comprise, for example, combiner 400 determining a mean and/or standard deviation (and/or variance) of probabilities in the selected subset. Combiner 400 generating a combined prediction 7300 may comprise determining a mean and/or standard deviation of aggregate predictions 7200. For example, combiner 400 may determine a mean 7210 and, optionally, a standard deviation (and/or variance) 7220 for each plurality of probabilities 7110 (and/or 7120) to generate each aggregate prediction 7200. Combiner 400 may further determine a mean of means 7210 to generate a mean 7310 of combined prediction 7310. Combiner 400 may further determine a standard deviation for mean 7310, e.g. by determining it directly from predictions 7100, based on standard deviations (and/or variances) 7220 and/or means 7210, and/or in any other suitable way. Combiner 400 may also, or alternatively, determine a confidence interval 7320 for prediction 450, e.g. in embodiments where prediction 450 comprises a probability of synergistic (and/or antagonistic) interaction. Confidence interval 450 may be determined in any suitable way, e.g. by propagation of uncertainties, and/or by assuming that mean 7310 of combined prediction 7300 is normally distributed and by determining a standard deviation and/or confidence interval 7320 based on standard deviations (and/or variances) 7220 and, if appropriate, a critical value and/or confidence level (which may, e.g., be predefined, user-provided, and/or otherwise obtained by combiner 400). In some implementations, system 1000 flags (i.e. identifies to a user) low-confidence predictions (i.e. candidate pesticidal compositions for which the confidence of a prediction is below a threshold) for experimental validation. Whether or not system 1000 performs such flagging, in some embodiments system 1000 is configured to re-train classifier 300 (via any suitable technique) on experimental results for such low-confidence predictions.

In some implementations, combiner 400 may generate aggregate predictions 7200 based on disjoint subsets of predictions 7100, e.g. as in the case where each aggregate prediction 7200 is generated from the predictions 7110 of a different classifier 310, as described above). In some implementations, combiner 400 generates predictions 7100 based on overlapping subsets of predictions 7100. For instance, combiner 400 may generate aggregate predictions convolutionally, e.g. by generating a first aggregate prediction based on a subset of predictions 7110 of a classifier 310 with iteration indices 1 through m (for some m<M) and generating a second aggregate prediction based on predictions 7110 of the same classifier 310 with iteration indices 2 through m+1.

FIG. 7 illustrates a data flow for an exemplary implementation of combiner 400. The combiner receives M predictions 7100 from each classifier 310 (parameterized by corresponding trained parameters 320). Predictions 7100 may be represented as a N×M matrix, where M is the number of iterations each trained classifier 310 performs, each resulting in a (potentially different) prediction 7100, e.g. of the probability of a synergistic interaction between the candidate compounds and/or a pest. N is the number of classifiers 310 that system 1000 is configured to use.

In at least that exemplary implementation, combiner 400 determines the mean and standard deviation (and/or variance) of the predictions 7100 for each iteration 1 . . . M. This is depicted in FIG. 7 as vectors of aggregate predictions 7200, and particularly as vectors of means 7210 and standard deviations (and/or variances) 7220. Combiner 400 determines a mean across aggregate predictions 7200, and particularly across means 7210, to generate a combined mean 7310 comprising a mean probability for the synergistic (and/or antagonistic) interaction. Combiner 400 optionally determines a confidence interval 7320 for combined mean 7310, e.g. by performing a propagation of uncertainty determination over standard deviations (and/or variances) 7220.

### Further Determinations Based on Synergy Predictions

In some embodiments, system 1000 generates prediction 450 by generating prediction 7300 as described above and providing prediction 7300 as prediction 450. In some embodiments (e.g. at least some of those without a combiner 400), system 1000 generates prediction 450 by providing at least one of the one or more predictions generated by classifier 300 (e.g. predictions 7100) as prediction 450. In some embodiments, system 1000 generates a prediction 450 by further transforming one or more of predictions 7100, 7200, and/or 7300. Such further transformations may be performed by combiner 400 and/or a post-processing module of system 1000 (not shown). In some embodiments, system 1000 generates a plurality of predictions 450, each in any of the foregoing ways. For instance, system 1000 may generate a first prediction 450 by providing prediction 7300 and may generate one or more further predictions 450 based on first prediction 450, one or more previously-generated further predictions 450, and/or one or more of predictions 7100, 7200, and/or 7300. For convenience, when discussing system 1000 generating a prediction 450 based on first prediction 450, one or more previously-generated further predictions 450, and/or one or more of predictions 7100, 7200, and/or 7300, such predictions (based on which a prediction 450 is generated) are referred to collectively and individually as “raw predictions”.

System 1000 may determine a prediction 450 in any of a variety of ways. In some embodiments, system 1000 generates a discretized prediction (such as a binary YES/NO or a categorical 1/2/3/4/5) based on one or more raw predictions being above or below one or more thresholds. For example, system 1000 may receive a threshold value (e.g. from a parameter store) and compare the threshold value to a raw prediction. If the threshold value is greater than (or, in some embodiments, no less than) the raw prediction, system 1000 may generate a discretized prediction with a value of TRUE, otherwise system 1000 may generate a discretized prediction with a value of FALSE.

In some embodiments, system 1000 generates a prediction 450 representing a predicted probability of a synergistic (and/or antagonistic) interaction existing between compounds of a candidate pesticidal composition and/or between one or more compounds of the candidate pesticidal composition and one or more pests based on one or more raw predictions. Alternatively, or in addition, system 1000 generates a prediction 450 representing a predicted degree of such synergistic (and/or antagonistic) interaction based on one or more raw predictions. Such a predicted degree may comprise a continuous-valued (e.g. floating-point) metric characterizing the predicted synergistic behavior of the candidate pesticidal composition. Such a predicted degree may comprise, for example, an order of magnitude of such a metric the synergistic interaction, e.g. determined by system 1000 based on a logarithm of the metric (e.g. log₂). In some embodiments, system 1000 generates a prediction 450 representing a value of a known synergy metric such as fractional inhibitory concentration index (FICI) and/or any other suitable metric, such as those disclosed by Greco, W. R., Bravo, G. & Parsons, J. C. (199). The search for synergy: a critical review from a response surface perspective. Pharmacological Reviews 47, 331-85.

In at least one example embodiment, system 1000 generates a prediction 450 representing a predicted degree of synergistic interaction comprising an order of magnitude of a synergy metric and maps the order of magnitude to a result based on one or more discretization criteria. For example, the discretization criteria may comprise configured level-of-effect bin threshold values and corresponding result values (e.g. obtained from a parameter store). System 1000 may compare the obtained level-of-effect bin threshold values to a value of the order of magnitude and thereby determine which result value to map the order of magnitude value to. For example, exemplary level-of-effect bin threshold values and corresponding result values are shown in the table below.

| Metric lower bound | Metric upper bound | Result |
|---|---|---|
| 0 | 2 | NONE |
| 2.01 | 4 | SLIGHT |
| 4.01 | 99.99 | STRONG |

Based on the threshold values and result values depicted in the above table, if the order of magnitude value is between 0 and 2, system 1000 maps the predicted degree of synergistic interaction to NONE. Similarly, if the order of magnitude value is greater than 2 and less than or equal to 4, system 1000 maps the predicted degree of synergistic interaction to “SLIGHT”, and if the order of magnitude value is greater than 4, system 1000 maps the predicted degree of synergistic interaction to “STRONG”. (Optionally, one or both of the top and bottom bounds, i.e. the 0 and 99.99 bounds, may instead be unbounded, such that any value less than 2 or greater than 4, respectively, would be mapped by system 1000 to the bin).

In some embodiments, system 1000 generates a prediction 450 comprising a predicted metric of effectiveness of the candidate pesticidal composition on one or more pests. System 1000 may determine the predicted metric of effectiveness by determining an amount of candidate pesticidal composition (e.g. the least amount predicted to be necessary) which provides effectiveness in vitro, in planta, and/or in field. Determining effectiveness in a pesticidal context may comprise determining that the composition (e.g. in a given amount) is predicted to suppress and/or control a pest population to within a threshold—example thresholds include achieving at least 90% mortality of a population of bedbugs in laboratory conditions. (A different threshold, such as 80%, 95%, or even 100%, may be used.) System 1000 may further combine the amount of candidate pesticidal composition with a per-unit resource allocation (e.g. as described above, such as by multiplication), such as a per-unit cost, to determine a predicted cost of efficacy metric for the candidate pesticidal composition.

System 1000 may output representations of the candidate pesticidal compositions for which predictions 450 are generated, which may comprise any of the representations of candidate pesticidal compositions described elsewhere herein and optionally also any of predictions 450, 7100, 7200, and/or 7300, and/or other information related to the candidate pesticidal compositions (collectively and individually the “output representations”). System 1000 may filter, rank, or otherwise modify the output representations of the candidate pesticidal compositions, e.g. based on any of the predictions 450, 7100, 7200, 7300, and/or other information related to the candidate pesticidal compositions.

For example, system 1000 may filter and/or rank candidate pesticidal compositions based on the cost of efficacy metric described above. System 1000 may identify the candidate pesticidal composition with the lowest cost of efficacy metric, a set of n candidate pesticidal compositions with the n lowest cost of efficacy metrics (for some value n, which may be predetermined, provided by a user, and/or otherwise obtained), a set of candidate pesticidal compositions with cost of efficacy metrics less than (or greater than) a threshold, and/or another set of one or more candidate pesticidal compositions based on their corresponding predicted metrics of effectiveness.

As another example, system 1000 may filter and/or rank candidate pesticidal compositions based on a predicted probability and/or degree of synergistic (and/or antagonistic) interaction of prediction 450. For example, system 1000 may determine that the probability (and/or degree) of such interaction for a given candidate pesticidal composition is less than (or greater than, no less than, or no greater than) a threshold value and may remove the candidate pesticidal composition and associated information from the output representations. System 1000 may alternatively, or additionally, rank the candidate pesticidal compositions of the output representations by such probability (e.g. from highest-probability to lowest-probability) and/or degree. Output representations may thus, for example, be limited to candidate pesticidal compositions which are predicted to be sufficiently likely to exhibit synergy (and/or predicted to exhibit synergy of sufficient degree) to warrant further testing. (Sufficiency here may be defined by the threshold, which may be predetermined, provided by a user, and/or otherwise obtained.)

As an illustrative example, system 1000 may remove candidate pesticidal compositions for which the corresponding prediction 450 indicates a <20% probability of a synergistic (and/or antagonistic) interaction. System 1000 may rank the remaining candidate pesticidal compositions from highest-probability to lowest-probability. Alternatively, or in addition, system 1000 may rank candidate pesticidal compositions for which the corresponding prediction 450 indicates a >80% probability of a synergistic (and/or antagonistic) interaction more highly than other candidate pesticidal compositions. It more highly ranks those results that have an approximately >80% probability of synergistic outcomes.

In some embodiments, system 1000 re-trains parameters 320 by comparing predictions 450 against results of laboratory and/or field tests and updating parameters 320 based on such comparisons (e.g. via active learning, online learning, and/or any other suitable technique). For example, system 1000 may update parameters 320 to minimize (or maximize, as appropriate) an objective function based on a difference between predictions 450 and test results. System 1000 may, for instance, perform gradient descent over the objective function based on the test results.

### Computer System

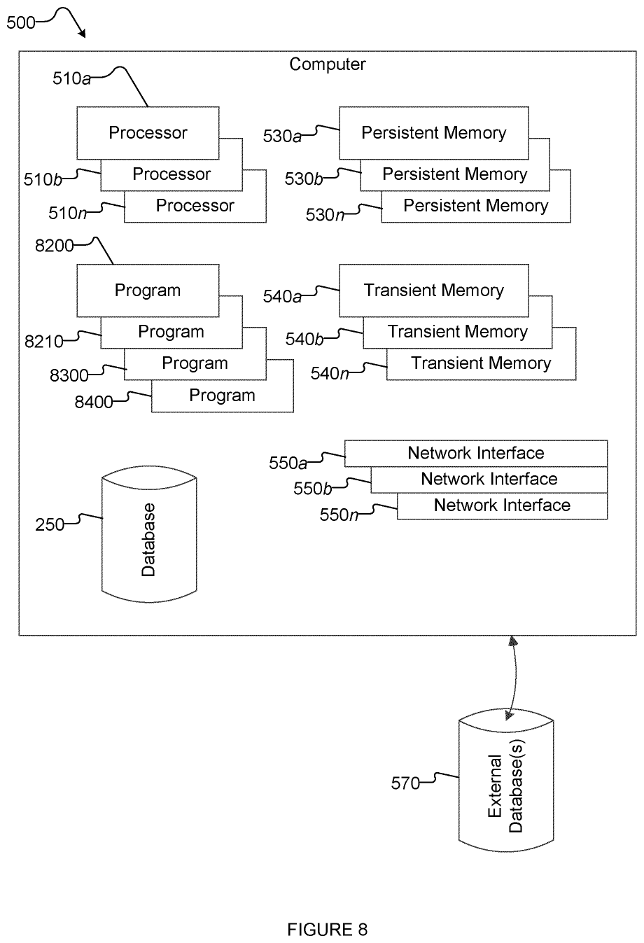

FIG. 8 illustrates an exemplary computer system providing system 1000. Each exemplary computer 500 comprises one or more processors 510a, . . . , 510n (collectively and individually processors 510) such as general purpose CPUs and/or specialty processors such as FPGAs or GPUs, operably connected to persistent memory 530 and/or transient memory 540 which store information being processed by system 1000 and may store executable instructions (collectively referred to herein as “programs”) (e.g. programs 8200, 8210, 8300, 8400 that perform the acts associated with like elements of system 1000, with reference numerals incremented by 8000) that executable by processors 510 to perform the methods described herein. Programs are described in more detail below. In some cases, such as FPGAs, programs comprise configuration information used to adapt processors 510 for particular purposes. One or more processors 510 may be operably connected to networking and communications interfaces 550 appropriate to the deployed configuration. Stored within persistent memories 530 of computers 500 may be one or more databases 250 used for the storage of information collected and/or calculated by the servers and read, processed, and written by processors 510 under control of program(s) (e.g. 8200, 8210, 8300, 8400). A computer 500 may also or alternatively be operably connected to an external database 570 via networking and communications interfaces 550.

Persistent memories 530 may include disk, PROM, EEPROM, flash storage, and similar technologies characterized by their ability to retain their contents between on/off power cycling of computer 500. Some persistent memories 530 may take the form of a file system for computer 500, and may be used to store control and operating programs and information that defines the manner in which computer 500 operates, including scheduling of background and foreground processes, as well as periodically performed processes. Persistent memories 530 in the form of network attached storage (NAS) (storage that is accessible over a network interface) may also or alternatively be used without departing from the scope of the disclosure. Transient memories 540 may include Random Access Memory (RAM) and similar technologies characterized by the contents of the storage not being retained between on/off power cycling of the system.

One or more databases 250, 570 may include local file storage, where the file system comprises the data storage and indexing scheme, a relational database, an object oriented database, an object relational database, a NOSQL database, and/or other database structures such as indexed record structures. Such databases 250 and/or 570 may be stored within a single persistent memory 530, may be stored across one or more persistent memories 530, and/or may be stored in persistent memories 530 on different computers.

System 1000 is illustrated with multiple logical databases for clarity. System 1000 may be deployed using one or more physical databases implemented on one or more computers 500, and/or on a virtualized computer system, and/or may be implemented using clustering techniques (e.g. so that at least a part of the data stored in a database is physically stored on two or more computers 500). In some implementations, one or more logical and/or physical databases may be implemented on a remote device and accessed over a communications network.

System 1000 further comprises several programs as described above (e.g. the above-described modules may be provided by programs of one or more computers 500).

Experimental Evaluation of Predictions and Formulation of Pesticidal Compositions and their Uses

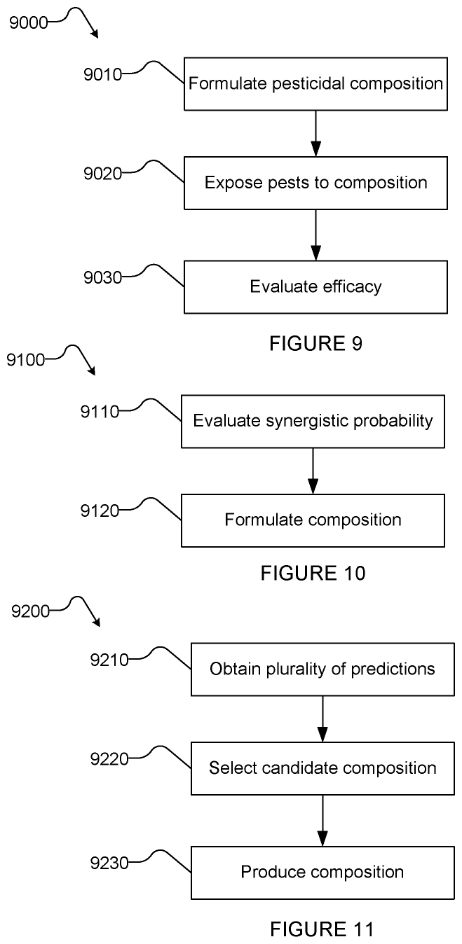

Once a prediction 450 has been determined, the results of that prediction can be used in any desired manner. For example, in one example method 9000 illustrated in FIG. 9, the prediction 450 can be evaluated against one or more pests in a test environment, for example in vivo or in planta, by formulating a composition containing the candidate pesticidal composition at 9010 (for example, by combining the pesticidal compound, the synergistic compound, and any desired formulation components such as solvents, carriers, adjuvants, stabilizers or the like) and exposing the one or more pests to the composition at 9020. At 9030, the efficacy of the composition as a pesticide is determined (for example by evaluating the efficacy of the composition in controlling or killing the one or more pests by assessing a percentage mortality of the pests and/or by assessing the time taken to reach peak mortality).

As another example, in method 9100 illustrated in FIG. 10, the prediction 450 can be used to formulate a pesticidal composition. At 9110, it is determined whether or not prediction 450 meets or exceeds a predetermined level of probability of a synergistic interaction, for example to determine whether there is a high probability that the candidate pesticidal composition containing the pesticidal compound and the synergistic compound is likely to exhibit a synergistic interaction against one or more pests. If prediction 450 meets or exceeds the predetermined level of probability of a synergistic interaction, then at 9120 a pesticidal composition containing the pesticidal compound and the synergistic compound and any desired formulation components such as solvents, carriers, adjuvants, stabilizers or the like is formulated.

As another example, in method 9200 illustrated in FIG. 11, the prediction 450 can be used to manufacture a pesticidal composition. At 9210, a plurality of predictions 450 of a synergistic interaction between a plurality of pesticidal compounds and a plurality of synergistic compounds are determined. Each prediction 450 corresponds to a proposed candidate pesticidal composition containing at least one pesticidal compound and at least one synergistic compound. At 9220, the plurality of predictions are evaluated and one proposed candidate pesticidal composition is selected based on desired characteristics of predictions 450. For example, a proposed candidate pesticidal composition with a prediction 450 that meets or exceeds a predetermined level of probability of being a synergistic interaction may be selected at 9220. Or a proposed candidate pesticidal composition with a prediction 450 that is higher than at least some of the other predictions 450 for other proposed candidate pesticidal compositions may be selected at 9220. The candidate pesticidal composition selected at 9220 is produced at step 9230, for example by mixing the pesticidal compound and the synergistic compound that make up the candidate pesticidal composition, together with any desired formulation components such as solvents, carriers, adjuvants, stabilizers or the like.

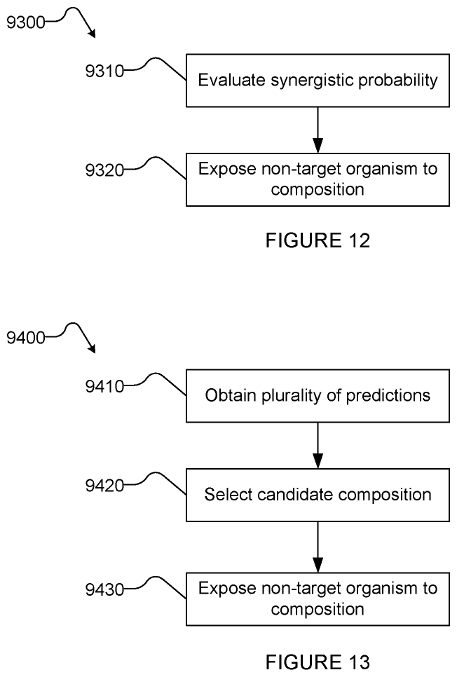

As another example, in method 9300 illustrated in FIG. 12, prediction 450 can be used to treat one or more pests affecting a non-target organism. At 9310, it is determined whether or not prediction 450 meets or exceeds a predetermined level of probability of a synergistic interaction, for example to determine whether there is a high probability that the candidate pesticidal composition containing the pesticidal compound and the synergistic compound is likely to exhibit a synergistic interaction against one or more pests. If prediction 450 meets or exceeds the predetermined level of probability of a synergistic interaction, then at 9320 the non-target organism can be exposed to a pesticidal composition that contains the candidate pesticidal composition. This will result in exposure of the one or more pests affecting the non-target organism to the pesticidal composition, to ameliorate or eliminate the adverse effects the one or more pests may have on the non-target organism.

As another example, in method 9400 illustrated in FIG. 13, prediction 450 can be used to treat one or more pests affecting a non-target organism. At 9410, a plurality of predictions 450 of a synergistic interaction between a plurality of pesticidal compounds and a plurality of synergistic compounds are determined. Each prediction 450 corresponds to a proposed candidate pesticidal composition containing at least one pesticidal compound and at least one synergistic compound. At 9420, the plurality of predictions are evaluated and one proposed candidate pesticidal composition is selected based on desired characteristics of predictions 450. For example, a proposed candidate pesticidal composition with a prediction 450 that meets or exceeds a predetermined level of probability of being a synergistic interaction may be selected at 9420. Or a proposed candidate pesticidal composition with a prediction 450 that is higher than at least some of the other predictions 450 for other proposed candidate pesticidal compositions may be selected at 9420. At step 9430, the non-target organism is exposed to a pesticidal composition containing the candidate pesticidal composition selected at 9420. This will result in exposure of the one or more pests affecting the non-target organism to the pesticidal composition, to ameliorate or eliminate the adverse effects the one or more pests may have on the non-target organism.

### Example Results

An implementation of system 1000 was used to generate predictions of the probability of existence of synergistic interactions between pairs of compounds in a set of candidate pesticidal compositions. For each prediction, system 1000 received representations of a pesticidal active compound and a potentially-synergistic compound. These representations of compounds were received as SMILES strings and enhanced via QSAR to produce a feature vector. (In some tests, the enhanced representation comprised graph representations of the compounds.) Features selected for consideration by this implementation of system 1000 included aromaticity, electronegativity, polarity, hydrophilicity/hydrophobicity, and hybridizations. System 1000 comprised three classifiers 310, each trained on synergistic efficacy of pesticidal compositions when applied against a different pest; pest information was not provided to classifiers 310 at inference time. The encoder was trained on a general chemistry dataset, namely Tox21. This implementation did not receive information on mixture ratios.

Laboratory experiments comprising in vitro testing of a pest treated with a candidate pesticidal composition (comprising the pesticidal and potentially-synergistic compounds) for each prediction were conducted to assess the accuracy of the predictions generated by the particular tested implementation of system 1000. Accuracy was assessed by determining the change in minimum inhibitory concentration (MIC) observed for each candidate pesticidal composition against the corresponding pest relative to the pesticidal compound without the potentially-synergistic compound. (The particular tested implementation comprised an ensemble classifier 300 and a combiner 400 which operated in accordance with the exemplary embodiment of FIG. 3.)

The tests encompassed six pesticidal active compounds and three fungal pests. Each of the pesticidal compounds was selected from a class known to have pesticidal effects against at least one of the three pests. They are identified below as Compounds A-F, and the pests are identified below as Pests A-C.

The potentially-synergistic compounds were selected from the group consisting of: C4-C10 unsaturated aliphatic acids: 10-hydroxydecanoic acid, 12-hydroxydodecanoic acid, 2,2-diethylbutanoic acid, 2-aminobutyric acid, 2-aminohexanoic acid, 2-ethylhexanoic acid, 2-hydroxybutyric acid, 2-hydroxyoctanoic acid, 2-methyldecanoic acid, 2-methyloctanoic acid, 3-aminobutyric acid, 3-decenoic acid, 3-heptenoic acid, 3-hydroxybutyric acid, 3-hydroxyhexanoic acid, 3-hydroxyoctanoic acid, 3-methylbutyric acid, 3-methylnonanoic acid, 3-nonenoic acid, 3-octenoic acid, 4-hexenoic acid, 4-methylhexanoic acid, 5-hexenoic acid, 7-octenoic acid, 8-hydroxyoctanoic acid, 9-decenoic acid, decanoic acid, dodecanoic acid, heptanoic acid, nonanoic acid, octanoic acid, oleic acid, sorbic acid, trans-2-nonenoic acid, trans-2-octenoic acid, trans-2-undecenoic acid, trans-3-hexenoic acid.

The tested implementation of system 1000 generated a prediction of a probability of the existence of a synergistic interaction between the compounds of each candidate pesticidal composition against each selected pest. As described above, the prediction of system 1000 was discretized such that probabilities less than or equal to 0.5 (i.e. 50%) were mapped to 0 (indicating no predicted synergy) and probabilities greater than 0.5 were mapped to 1 (indicating a predicted synergy). The binarized results are presented in Table 1 under the “Prediction” column. In Table 1, the value of the prediction column is the discretized prediction of system 1000. The value of the “Observation” column is the result observed in the above-described laboratory experiments, expressed as degree of synergy (in this instance, inverse FICI). For example, a value of 4 means that the observed FICI value was ¼. Values greater than 1 are synergistic.

TABLE 1
Results of pair-wise synergy prediction tests on selected pest organisms.

| Pest | Pesticidal Compound | Synergistic Compound | Prediction | Observation |
|---|---|---|---|---|
| Pest-A | Compound-A | 10-hydroxydecanoic acid | 0 | 1 |
| Pest-B | Compound-A | 10-hydroxydecanoic acid | 1 | 4 |
| Pest-A | Compound-B | 10-hydroxydecanoic acid | 0 | 1 |
| Pest-B | Compound-B | 10-hydroxydecanoic acid | 0 | 1 |
| Pest-C | Compound-B | 10-hydroxydecanoic acid | 1 | 4 |
| Pest-C | Compound-C | 10-hydroxydecanoic acid | 1 | 4 |
| Pest-B | Compound-D | 10-hydroxydecanoic acid | 0 | 1 |
| Pest-C | Compound-D | 10-hydroxydecanoic acid | 1 | 4 |
| Pest-A | Compound-E | 10-hydroxydecanoic acid | 0 | 1 |
| Pest-C | Compound-E | 10-hydroxydecanoic acid | 1 | 4 |
| Pest-B | Compound-E | 10-hydroxydecanoic acid | 1 | 4 |
| Pest-B | Compound-F | 10-hydroxydecanoic acid | 0 | 1 |
| Pest-A | Compound-F | 10-hydroxydecanoic acid | 1 | 4 |
| Pest-C | Compound-F | 10-hydroxydecanoic acid | 1 | 4 |
| Pest-B | Compound-A | 12-hydroxydodecanoic acid | 0 | 1 |
| Pest-A | Compound-B | 12-hydroxydodecanoic acid | 0 | 1 |
| Pest-B | Compound-B | 12-hydroxydodecanoic acid | 1 | 4 |
| Pest-B | Compound-D | 12-hydroxydodecanoic acid | 0 | 1 |
| Pest-A | Compound-E | 12-hydroxydodecanoic acid | 1 | 4 |
| Pest-B | Compound-E | 12-hydroxydodecanoic acid | 1 | 4 |
| Pest-A | Compound-F | 12-hydroxydodecanoic acid | 0 | 1 |
| Pest-B | Compound-F | 12-hydroxydodecanoic acid | 0 | 1 |
| Pest-A | Compound-A | 2,2-diethylbutanoic acid | 0 | 1 |
| Pest-B | Compound-A | 2,2-diethylbutanoic acid | 0 | 1 |
| Pest-C | Compound-B | 2,2-diethylbutanoic acid | 0 | 1 |
| Pest-B | Compound-B | 2,2-diethylbutanoic acid | 0 | 1 |
| Pest-A | Compound-B | 2,2-diethylbutanoic acid | 1 | 4 |
| Pest-C | Compound-C | 2,2-diethylbutanoic acid | 0 | 1 |
| Pest-C | Compound-D | 2,2-diethylbutanoic acid | 0 | 1 |
| Pest-B | Compound-D | 2,2-diethylbutanoic acid | 1 | 4 |
| Pest-A | Compound-E | 2,2-diethylbutanoic acid | 0 | 1 |
| Pest-C | Compound-E | 2,2-diethylbutanoic acid | 0 | 1 |
| Pest-B | Compound-E | 2,2-diethylbutanoic acid | 1 | 4 |
| Pest-C | Compound-F | 2,2-diethylbutanoic acid | 0 | 1 |
| Pest-A | Compound-F | 2,2-diethylbutanoic acid | 1 | 4 |
| Pest-B | Compound-F | 2,2-diethylbutanoic acid | 1 | 4 |
| Pest-C | Compound-E | 2-aminobutyric acid | 0 | 1 |
| Pest-C | Compound-F | 2-aminobutyric acid | 0 | 1 |
| Pest-C | Compound-B | 2-aminohexanoic acid | 1 | 4 |
| Pest-C | Compound-C | 2-aminohexanoic acid | 1 | 4 |
| Pest-C | Compound-D | 2-aminohexanoic acid | 1 | 4 |
| Pest-C | Compound-E | 2-aminohexanoic acid | 1 | 4 |
| Pest-C | Compound-F | 2-aminohexanoic acid | 1 | 4 |
| Pest-A | Compound-A | 2-ethylhexanoic acid | 0 | 1 |
| Pest-B | Compound-A | 2-ethylhexanoic acid | 1 | 4 |
| Pest-A | Compound-B | 2-ethylhexanoic acid | 0 | 1 |
| Pest-C | Compound-B | 2-ethylhexanoic acid | 0 | 1 |
| Pest-B | Compound-B | 2-ethylhexanoic acid | 0 | 1 |
| Pest-C | Compound-C | 2-ethylhexanoic acid | 0 | 1 |
| Pest-A | Compound-D | 2-ethylhexanoic acid | 0 | 1 |
| Pest-C | Compound-D | 2-ethylhexanoic acid | 0 | 1 |
| Pest-B | Compound-D | 2-ethylhexanoic acid | 1 | 4 |
| Pest-C | Compound-E | 2-ethylhexanoic acid | 0 | 1 |
| Pest-A | Compound-E | 2-ethylhexanoic acid | 1 | 4 |
| Pest-B | Compound-E | 2-ethylhexanoic acid | 1 | 4 |
| Pest-C | Compound-F | 2-ethylhexanoic acid | 0 | 1 |
| Pest-A | Compound-F | 2-ethylhexanoic acid | 1 | 4 |
| Pest-B | Compound-F | 2-ethylhexanoic acid | 1 | 4 |
| Pest-B | Compound-A | 2-hydroxybutyric acid | 0 | 1 |
| Pest-B | Compound-B | 2-hydroxybutyric acid | 0 | 1 |
| Pest-A | Compound-B | 2-hydroxybutyric acid | 1 | 4 |
| Pest-C | Compound-C | 2-hydroxybutyric acid | 1 | 4 |
| Pest-A | Compound-D | 2-hydroxybutyric acid | 0 | 1 |
| Pest-B | Compound-D | 2-hydroxybutyric acid | 0 | 1 |
| Pest-C | Compound-D | 2-hydroxybutyric acid | 1 | 4 |
| Pest-A | Compound-E | 2-hydroxybutyric acid | 0 | 1 |
| Pest-B | Compound-E | 2-hydroxybutyric acid | 0 | 1 |
| Pest-C | Compound-E | 2-hydroxybutyric acid | 1 | 4 |
| Pest-C | Compound-F | 2-hydroxybutyric acid | 0 | 1 |
| Pest-B | Compound-F | 2-hydroxybutyric acid | 0 | 1 |
| Pest-B | Compound-A | 2-hydroxyhexanoic acid | 1 | 4 |
| Pest-A | Compound-B | 2-hydroxyhexanoic acid | 0 | 1 |
| Pest-B | Compound-B | 2-hydroxyhexanoic acid | 1 | 4 |
| Pest-C | Compound-C | 2-hydroxyhexanoic acid | 1 | 4 |
| Pest-C | Compound-D | 2-hydroxyhexanoic acid | 1 | 4 |
| Pest-B | Compound-D | 2-hydroxyhexanoic acid | 1 | 4 |
| Pest-A | Compound-E | 2-hydroxyhexanoic acid | 0 | 1 |
| Pest-C | Compound-E | 2-hydroxyhexanoic acid | 1 | 4 |
| Pest-B | Compound-E | 2-hydroxyhexanoic acid | 1 | 4 |
| Pest-A | Compound-F | 2-hydroxyhexanoic acid | 1 | 4 |
| Pest-C | Compound-F | 2-hydroxyhexanoic acid | 1 | 4 |
| Pest-B | Compound-F | 2-hydroxyhexanoic acid | 1 | 4 |
| Pest-B | Compound-A | 2-hydroxyoctanoic acid | 0 | 1 |
| Pest-C | Compound-B | 2-hydroxyoctanoic acid | 0 | 1 |
| Pest-B | Compound-B | 2-hydroxyoctanoic acid | 0 | 1 |
| Pest-C | Compound-C | 2-hydroxyoctanoic acid | 0 | 1 |
| Pest-B | Compound-D | 2-hydroxyoctanoic acid | 0 | 1 |
| Pest-C | Compound-D | 2-hydroxyoctanoic acid | 1 | 4 |
| Pest-C | Compound-E | 2-hydroxyoctanoic acid | 0 | 1 |
| Pest-B | Compound-E | 2-hydroxyoctanoic acid | 0 | 1 |
| Pest-C | Compound-F | 2-hydroxyoctanoic acid | 0 | 1 |
| Pest-B | Compound-F | 2-hydroxyoctanoic acid | 0 | 1 |
| Pest-A | Compound-A | 2-methyldecanoic acid | 0 | 1 |
| Pest-A | Compound-B | 2-methyldecanoic acid | 0 | 1 |
| Pest-C | Compound-B | 2-methyldecanoic acid | 0 | 1 |
| Pest-B | Compound-B | 2-methyldecanoic acid | 0 | 1 |
| Pest-C | Compound-C | 2-methyldecanoic acid | 0 | 1 |
| Pest-C | Compound-D | 2-methyldecanoic acid | 0 | 1 |
| Pest-B | Compound-D | 2-methyldecanoic acid | 0 | 1 |
| Pest-A | Compound-E | 2-methyldecanoic acid | 0 | 1 |
| Pest-C | Compound-E | 2-methyldecanoic acid | 0 | 1 |
| Pest-B | Compound-E | 2-methyldecanoic acid | 1 | 4 |
| Pest-B | Compound-F | 2-methyldecanoic acid | 0 | 1 |
| Pest-A | Compound-F | 2-methyldecanoic acid | 1 | 4 |
| Pest-C | Compound-F | 2-methyldecanoic acid | 1 | 4 |
| Pest-A | Compound-A | 2-methyloctanoic acid | 0 | 1 |
| Pest-B | Compound-A | 2-methyloctanoic acid | 1 | 4 |
| Pest-C | Compound-B | 2-methyloctanoic acid | 0 | 1 |
| Pest-A | Compound-B | 2-methyloctanoic acid | 1 | 4 |
| Pest-C | Compound-C | 2-methyloctanoic acid | 0 | 1 |
| Pest-C | Compound-D | 2-methyloctanoic acid | 0 | 1 |
| Pest-B | Compound-D | 2-methyloctanoic acid | 1 | 4 |
| Pest-A | Compound-E | 2-methyloctanoic acid | 0 | 1 |
| Pest-C | Compound-E | 2-methyloctanoic acid | 0 | 1 |
| Pest-B | Compound-E | 2-methyloctanoic acid | 1 | 4 |
| Pest-C | Compound-F | 2-methyloctanoic acid | 0 | 1 |
| Pest-B | Compound-F | 2-methyloctanoic acid | 0 | 1 |
| Pest-A | Compound-F | 2-methyloctanoic acid | 1 | 4 |
| Pest-A | Compound-A | 3-aminobutyric acid | 0 | 1 |
| Pest-A | Compound-B | 3-aminobutyric acid | 0 | 1 |
| Pest-C | Compound-B | 3-aminobutyric acid | 1 | 4 |
| Pest-C | Compound-C | 3-aminobutyric acid | 1 | 4 |
| Pest-C | Compound-D | 3-aminobutyric acid | 1 | 4 |
| Pest-A | Compound-E | 3-aminobutyric acid | 1 | 4 |
| Pest-C | Compound-E | 3-aminobutyric acid | 1 | 4 |
| Pest-C | Compound-F | 3-aminobutyric acid | 1 | 4 |
| Pest-A | Compound-A | 3-decenoic acid | 1 | 8 |
| Pest-C | Compound-A | 3-decenoic acid | 1 | 8 |
| Pest-B | Compound-A | 3-decenoic acid | 1 | 12 |
| Pest-A | Compound-G | 3-decenoic acid | 0 | 1 |
| Pest-C | Compound-G | 3-decenoic acid | 0 | 1 |
| Pest-B | Compound-G | 3-decenoic acid | 0 | 1 |
| Pest-A | Compound-B | 3-decenoic acid | 0 | 1 |
| Pest-B | Compound-B | 3-decenoic acid | 1 | 4 |
| Pest-A | Compound-C | 3-decenoic acid | 1 | 16 |
| Pest-C | Compound-C | 3-decenoic acid | 1 | 4 |
| Pest-B | Compound-C | 3-decenoic acid | 1 | 4 |
| Pest-A | Compound-D | 3-decenoic acid | 0 | 1 |
| Pest-B | Compound-D | 3-decenoic acid | 0 | 1 |
| Pest-C | Compound-D | 3-decenoic acid | 1 | 4 |
| Pest-A | Compound-H | 3-decenoic acid | 0 | 1 |
| Pest-A | Compound-I | 3-decenoic acid | 0 | 1 |
| Pest-C | Compound-I | 3-decenoic acid | 0 | 1 |
| Pest-B | Compound-I | 3-decenoic acid | 0 | 1 |
| Pest-A | Compound-E | 3-decenoic acid | 1 | 8 |
| Pest-C | Compound-E | 3-decenoic acid | 1 | 4 |
| Pest-B | Compound-E | 3-decenoic acid | 1 | 32 |
| Pest-C | Compound-F | 3-decenoic acid | 0 | 1 |
| Pest-A | Compound-F | 3-decenoic acid | 1 | 4 |
| Pest-B | Compound-F | 3-decenoic acid | 1 | 4 |
| Pest-A | Compound-A | 3-heptenoic acid | 0 | 1 |
| Pest-C | Compound-A | 3-heptenoic acid | 1 | 8 |
| Pest-B | Compound-A | 3-heptenoic acid | 1 | 4 |
| Pest-A | Compound-G | 3-heptenoic acid | 0 | 1 |
| Pest-C | Compound-G | 3-heptenoic acid | 0 | 1 |
| Pest-B | Compound-G | 3-heptenoic acid | 0 | 1 |
| Pest-A | Compound-B | 3-heptenoic acid | 0 | 1 |
| Pest-B | Compound-B | 3-heptenoic acid | 0 | 1 |
| Pest-B | Compound-C | 3-heptenoic acid | 0 | 1 |
| Pest-A | Compound-C | 3-heptenoic acid | 1 | 4 |
| Pest-C | Compound-C | 3-heptenoic acid | 1 | 4 |
| Pest-A | Compound-D | 3-heptenoic acid | 0 | 1 |
| Pest-B | Compound-D | 3-heptenoic acid | 0 | 1 |
| Pest-C | Compound-D | 3-heptenoic acid | 1 | 4 |
| Pest-A | Compound-H | 3-heptenoic acid | 0 | 1 |
| Pest-A | Compound-I | 3-heptenoic acid | 0 | 1 |
| Pest-C | Compound-I | 3-heptenoic acid | 0 | 1 |
| Pest-B | Compound-I | 3-heptenoic acid | 1 | 4 |
| Pest-A | Compound-E | 3-heptenoic acid | 1 | 4 |
| Pest-C | Compound-E | 3-heptenoic acid | 1 | 8 |
| Pest-B | Compound-E | 3-heptenoic acid | 1 | 8 |
| Pest-A | Compound-F | 3-heptenoic acid | 0 | 1 |
| Pest-C | Compound-F | 3-heptenoic acid | 0 | 1 |
| Pest-B | Compound-F | 3-heptenoic acid | 1 | 2 |
| Pest-A | Compound-A | 3-hydroxybutyric acid | 0 | 1 |
| Pest-B | Compound-A | 3-hydroxybutyric acid | 0 | 1 |
| Pest-C | Compound-G | 3-hydroxybutyric acid | 0 | 1 |
| Pest-A | Compound-G | 3-hydroxybutyric acid | 0 | 1 |
| Pest-C | Compound-G | 3-hydroxybutyric acid | 0 | 1 |
| Pest-B | Compound-G | 3-hydroxybutyric acid | 0 | 1 |
| Pest-A | Compound-J | 3-hydroxybutyric acid | 0 | 1 |
| Pest-B | Compound-J | 3-hydroxybutyric acid | 0 | 1 |
| Pest-C | Compound-B | 3-hydroxybutyric acid | 0 | 1 |
| Pest-A | Compound-B | 3-hydroxybutyric acid | 0 | 1 |
| Pest-B | Compound-B | 3-hydroxybutyric acid | 0 | 1 |
| Pest-C | Compound-B | 3-hydroxybutyric acid | 1 | 4 |
| Pest-A | Compound-C | 3-hydroxybutyric acid | 1 | 4 |
| Pest-C | Compound-C | 3-hydroxybutyric acid | 1 | 4 |
| Pest-B | Compound-C | 3-hydroxybutyric acid | 1 | 4 |
| Pest-C | Compound-D | 3-hydroxybutyric acid | 0 | 1 |
| Pest-A | Compound-D | 3-hydroxybutyric acid | 0 | 1 |
| Pest-B | Compound-D | 3-hydroxybutyric acid | 0 | 1 |
| Pest-C | Compound-D | 3-hydroxybutyric acid | 1 | 4 |
| Pest-A | Compound-H | 3-hydroxybutyric acid | 0 | 1 |
| Pest-B | Compound-H | 3-hydroxybutyric acid | 0 | 1 |
| Pest-C | Compound-I | 3-hydroxybutyric acid | 0 | 1 |
| Pest-A | Compound-I | 3-hydroxybutyric acid | 0 | 1 |
| Pest-B | Compound-I | 3-hydroxybutyric acid | 0 | 1 |
| Pest-C | Compound-E | 3-hydroxybutyric acid | 1 | 4 |
| Pest-B | Compound-E | 3-hydroxybutyric acid | 1 | 256 |
| Pest-A | Compound-E | 3-hydroxybutyric acid | 1 | 4 |
| Pest-C | Compound-E | 3-hydroxybutyric acid | 1 | 4 |
| Pest-B | Compound-E | 3-hydroxybutyric acid | 1 | 4 |
| Pest-A | Compound-F | 3-hydroxybutyric acid | 1 | 4 |
| Pest-C | Compound-F | 3-hydroxybutyric acid | 1 | 4 |
| Pest-B | Compound-F | 3-hydroxybutyric acid | 1 | 4 |
| Pest-A | Compound-A | 3-hydroxydecanoic acid | 0 | 1 |
| Pest-B | Compound-A | 3-hydroxydecanoic acid | 0 | 1 |
| Pest-A | Compound-G | 3-hydroxydecanoic acid | 0 | 1 |
| Pest-B | Compound-G | 3-hydroxydecanoic acid | 0 | 1 |
| Pest-C | Compound-G | 3-hydroxydecanoic acid | 1 | 16 |
| Pest-C | Compound-G | 3-hydroxydecanoic acid | 1 | 4 |
| Pest-A | Compound-J | 3-hydroxydecanoic acid | 0 | 1 |
| Pest-B | Compound-J | 3-hydroxydecanoic acid | 0 | 1 |
| Pest-C | Compound-J | 3-hydroxydecanoic acid | 1 | 4 |
| Pest-C | Compound-B | 3-hydroxydecanoic acid | 0 | 1 |
| Pest-A | Compound-B | 3-hydroxydecanoic acid | 0 | 1 |
| Pest-C | Compound-B | 3-hydroxydecanoic acid | 0 | 1 |
| Pest-B | Compound-B | 3-hydroxydecanoic acid | 0 | 1 |
| Pest-A | Compound-C | 3-hydroxydecanoic acid | 0 | 1 |
| Pest-C | Compound-C | 3-hydroxydecanoic acid | 0 | 1 |
| Pest-B | Compound-C | 3-hydroxydecanoic acid | 0 | 1 |
| Pest-A | Compound-D | 3-hydroxydecanoic acid | 0 | 1 |
| Pest-C | Compound-D | 3-hydroxydecanoic acid | 0 | 1 |
| Pest-B | Compound-D | 3-hydroxydecanoic acid | 0 | 1 |
| Pest-C | Compound-D | 3-hydroxydecanoic acid | 1 | 16 |
| Pest-A | Compound-H | 3-hydroxydecanoic acid | 0 | 1 |
| Pest-B | Compound-H | 3-hydroxydecanoic acid | 0 | 1 |
| Pest-C | Compound-I | 3-hydroxydecanoic acid | 0 | 1 |
| Pest-A | Compound-I | 3-hydroxydecanoic acid | 0 | 1 |
| Pest-B | Compound-I | 3-hydroxydecanoic acid | 0 | 1 |
| Pest-A | Compound-E | 3-hydroxydecanoic acid | 0 | 1 |
| Pest-C | Compound-E | 3-hydroxydecanoic acid | 1 | 16 |
| Pest-B | Compound-E | 3-hydroxydecanoic acid | 1 | 256 |
| Pest-C | Compound-E | 3-hydroxydecanoic acid | 1 | 4 |
| Pest-B | Compound-E | 3-hydroxydecanoic acid | 1 | 4 |
| Pest-A | Compound-F | 3-hydroxydecanoic acid | 0 | 1 |
| Pest-B | Compound-F | 3-hydroxydecanoic acid | 0 | 1 |
| Pest-C | Compound-F | 3-hydroxydecanoic acid | 1 | 4 |
| Pest-B | Compound-E | 3-hydroxyhexanoic acid | 1 | 512 |
| Pest-A | Compound-A | 3-hydroxyhexanoic acid | 0 | 1 |
| Pest-B | Compound-A | 3-hydroxyhexanoic acid | 1 | 4 |
| Pest-A | Compound-G | 3-hydroxyhexanoic acid | 0 | 1 |
| Pest-C | Compound-G | 3-hydroxyhexanoic acid | 0 | 1 |
| Pest-B | Compound-G | 3-hydroxyhexanoic acid | 0 | 1 |
| Pest-A | Compound-J | 3-hydroxyhexanoic acid | 0 | 1 |
| Pest-B | Compound-J | 3-hydroxyhexanoic acid | 0 | 1 |
| Pest-A | Compound-B | 3-hydroxyhexanoic acid | 0 | 1 |
| Pest-C | Compound-B | 3-hydroxyhexanoic acid | 0 | 1 |
| Pest-B | Compound-B | 3-hydroxyhexanoic acid | 0 | 1 |
| Pest-A | Compound-C | 3-hydroxyhexanoic acid | 0 | 1 |
| Pest-C | Compound-C | 3-hydroxyhexanoic acid | 1 | 4 |
| Pest-B | Compound-C | 3-hydroxyhexanoic acid | 1 | 4 |
| Pest-A | Compound-D | 3-hydroxyhexanoic acid | 0 | 1 |
| Pest-C | Compound-D | 3-hydroxyhexanoic acid | 0 | 1 |
| Pest-B | Compound-D | 3-hydroxyhexanoic acid | 0 | 1 |
| Pest-A | Compound-H | 3-hydroxyhexanoic acid | 0 | 1 |
| Pest-B | Compound-H | 3-hydroxyhexanoic acid | 0 | 1 |
| Pest-A | Compound-I | 3-hydroxyhexanoic acid | 0 | 1 |
| Pest-B | Compound-I | 3-hydroxyhexanoic acid | 0 | 1 |
| Pest-A | Compound-E | 3-hydroxyhexanoic acid | 0 | 1 |
| Pest-C | Compound-E | 3-hydroxyhexanoic acid | 0 | 1 |
| Pest-B | Compound-E | 3-hydroxyhexanoic acid | 1 | 4 |
| Pest-A | Compound-F | 3-hydroxyhexanoic acid | 0 | 1 |
| Pest-C | Compound-F | 3-hydroxyhexanoic acid | 0 | 1 |
| Pest-B | Compound-F | 3-hydroxyhexanoic acid | 1 | 4 |
| Pest-B | Compound-A | 3-hydroxyoctanoic acid | 1 | 4 |
| Pest-A | Compound-B | 3-hydroxyoctanoic acid | 0 | 1 |
| Pest-C | Compound-B | 3-hydroxyoctanoic acid | 0 | 1 |
| Pest-B | Compound-B | 3-hydroxyoctanoic acid | 0 | 1 |
| Pest-B | Compound-D | 3-hydroxyoctanoic acid | 0 | 1 |
| Pest-A | Compound-D | 3-hydroxyoctanoic acid | 1 | 4 |
| Pest-A | Compound-E | 3-hydroxyoctanoic acid | 1 | 4 |
| Pest-B | Compound-E | 3-hydroxyoctanoic acid | 1 | 4 |
| Pest-C | Compound-F | 3-hydroxyoctanoic acid | 0 | 1 |
| Pest-B | Compound-F | 3-hydroxyoctanoic acid | 0 | 1 |
| Pest-A | Compound-F | 3-hydroxyoctanoic acid | 1 | 4 |
| Pest-A | Compound-A | 3-methylbutyric acid | 0 | 1 |
| Pest-B | Compound-A | 3-methylbutyric acid | 1 | 4 |
| Pest-C | Compound-B | 3-methylbutyric acid | 0 | 1 |
| Pest-B | Compound-B | 3-methylbutyric acid | 0 | 1 |
| Pest-A | Compound-B | 3-methylbutyric acid | 1 | 4 |
| Pest-C | Compound-C | 3-methylbutyric acid | 0 | 1 |
| Pest-A | Compound-D | 3-methylbutyric acid | 0 | 1 |
| Pest-B | Compound-D | 3-methylbutyric acid | 1 | 4 |
| Pest-A | Compound-E | 3-methylbutyric acid | 0 | 1 |
| Pest-B | Compound-E | 3-methylbutyric acid | 1 | 4 |
| Pest-C | Compound-F | 3-methylbutyric acid | 0 | 1 |
| Pest-A | Compound-F | 3-methylbutyric acid | 1 | 4 |
| Pest-B | Compound-F | 3-methylbutyric acid | 1 | 4 |
| Pest-B | Compound-A | 3-methylhexanoic acid | 0 | 1 |
| Pest-A | Compound-B | 3-methylhexanoic acid | 0 | 1 |
| Pest-B | Compound-B | 3-methylhexanoic acid | 0 | 1 |
| Pest-C | Compound-C | 3-methylhexanoic acid | 0 | 1 |
| Pest-C | Compound-E | 3-methylhexanoic acid | 0 | 1 |
| Pest-A | Compound-E | 3-methylhexanoic acid | 1 | 4 |
| Pest-B | Compound-E | 3-methylhexanoic acid | 1 | 4 |
| Pest-C | Compound-F | 3-methylhexanoic acid | 0 | 1 |
| Pest-B | Compound-F | 3-methylhexanoic acid | 0 | 1 |
| Pest-A | Compound-F | 3-methylhexanoic acid | 1 | 4 |
| Pest-A | Compound-A | 3-methylnonanoic acid | 0 | 1 |
| Pest-B | Compound-A | 3-methylnonanoic acid | 1 | 4 |
| Pest-A | Compound-B | 3-methylnonanoic acid | 0 | 1 |
| Pest-C | Compound-B | 3-methylnonanoic acid | 0 | 1 |
| Pest-B | Compound-B | 3-methylnonanoic acid | 0 | 1 |
| Pest-C | Compound-C | 3-methylnonanoic acid | 0 | 1 |
| Pest-C | Compound-D | 3-methylnonanoic acid | 0 | 1 |
| Pest-A | Compound-E | 3-methylnonanoic acid | 0 | 1 |
| Pest-C | Compound-E | 3-methylnonanoic acid | 0 | 1 |
| Pest-B | Compound-E | 3-methylnonanoic acid | 1 | 4 |
| Pest-C | Compound-F | 3-methylnonanoic acid | 0 | 1 |
| Pest-B | Compound-F | 3-methylnonanoic acid | 0 | 1 |
| Pest-C | Compound-A | 3-nonenoic acid | 0 | 1 |
| Pest-A | Compound-A | 3-nonenoic acid | 1 | 4 |
| Pest-B | Compound-A | 3-nonenoic acid | 1 | 10 |
| Pest-A | Compound-G | 3-nonenoic acid | 0 | 1 |
| Pest-C | Compound-G | 3-nonenoic acid | 0 | 1 |
| Pest-B | Compound-G | 3-nonenoic acid | 1 | 4 |
| Pest-A | Compound-B | 3-nonenoic acid | 0 | 1 |
| Pest-B | Compound-B | 3-nonenoic acid | 1 | 4 |
| Pest-C | Compound-C | 3-nonenoic acid | 0 | 1 |
| Pest-B | Compound-C | 3-nonenoic acid | 0 | 1 |
| Pest-A | Compound-C | 3-nonenoic acid | 1 | 8 |
| Pest-A | Compound-D | 3-nonenoic acid | 0 | 1 |
| Pest-C | Compound-D | 3-nonenoic acid | 0 | 1 |
| Pest-B | Compound-D | 3-nonenoic acid | 1 | 2 |
| Pest-A | Compound-H | 3-nonenoic acid | 1 | 4 |
| Pest-A | Compound-I | 3-nonenoic acid | 0 | 1 |
| Pest-C | Compound-I | 3-nonenoic acid | 0 | 1 |
| Pest-B | Compound-I | 3-nonenoic acid | 0 | 1 |
| Pest-C | Compound-E | 3-nonenoic acid | 0 | 1 |
| Pest-A | Compound-E | 3-nonenoic acid | 1 | 4 |
| Pest-B | Compound-E | 3-nonenoic acid | 1 | 16 |
| Pest-A | Compound-F | 3-nonenoic acid | 0 | 1 |
| Pest-C | Compound-F | 3-nonenoic acid | 1 | 4 |
| Pest-B | Compound-F | 3-nonenoic acid | 1 | 2 |
| Pest-C | Compound-A | 3-octenoic acid | 0 | 1 |
| Pest-A | Compound-A | 3-octenoic acid | 1 | 4 |
| Pest-B | Compound-A | 3-octenoic acid | 1 | 6 |
| Pest-A | Compound-G | 3-octenoic acid | 0 | 1 |
| Pest-C | Compound-G | 3-octenoic acid | 0 | 1 |
| Pest-B | Compound-G | 3-octenoic acid | 0 | 1 |
| Pest-A | Compound-B | 3-octenoic acid | 0 | 1 |
| Pest-B | Compound-B | 3-octenoic acid | 0 | 1 |
| Pest-C | Compound-B | 3-octenoic acid | 1 | 4 |
| Pest-B | Compound-C | 3-octenoic acid | 0 | 1 |
| Pest-A | Compound-C | 3-octenoic acid | 1 | 4 |
| Pest-C | Compound-C | 3-octenoic acid | 1 | 4 |
| Pest-A | Compound-D | 3-octenoic acid | 0 | 1 |
| Pest-C | Compound-D | 3-octenoic acid | 0 | 1 |
| Pest-B | Compound-D | 3-octenoic acid | 1 | 2 |
| Pest-A | Compound-H | 3-octenoic acid | 0 | 1 |
| Pest-A | Compound-I | 3-octenoic acid | 0 | 1 |
| Pest-C | Compound-I | 3-octenoic acid | 0 | 1 |
| Pest-B | Compound-I | 3-octenoic acid | 0 | 1 |
| Pest-C | Compound-E | 3-octenoic acid | 0 | 1 |
| Pest-A | Compound-E | 3-octenoic acid | 1 | 6 |
| Pest-B | Compound-E | 3-octenoic acid | 1 | 16 |
| Pest-A | Compound-F | 3-octenoic acid | 0 | 1 |
| Pest-B | Compound-F | 3-octenoic acid | 0 | 1 |
| Pest-C | Compound-F | 3-octenoic acid | 1 | 4 |
| Pest-A | Compound-A | 4-hexenoic acid | 1 | 2 |
| Pest-A | Compound-G | 4-hexenoic acid | 0 | 1 |
| Pest-C | Compound-G | 4-hexenoic acid | 0 | 1 |
| Pest-A | Compound-B | 4-hexenoic acid | 0 | 1 |
| Pest-B | Compound-B | 4-hexenoic acid | 0 | 1 |
| Pest-C | Compound-B | 4-hexenoic acid | 1 | 4 |
| Pest-B | Compound-C | 4-hexenoic acid | 0 | 1 |
| Pest-A | Compound-D | 4-hexenoic acid | 0 | 1 |
| Pest-C | Compound-D | 4-hexenoic acid | 0 | 1 |
| Pest-B | Compound-D | 4-hexenoic acid | 0 | 1 |
| Pest-A | Compound-H | 4-hexenoic acid | 0 | 1 |
| Pest-A | Compound-I | 4-hexenoic acid | 0 | 1 |
| Pest-C | Compound-I | 4-hexenoic acid | 0 | 1 |
| Pest-B | Compound-I | 4-hexenoic acid | 0 | 1 |
| Pest-A | Compound-E | 4-hexenoic acid | 1 | 2 |
| Pest-C | Compound-E | 4-hexenoic acid | 1 | 4 |
| Pest-B | Compound-E | 4-hexenoic acid | 1 | 8 |
| Pest-C | Compound-F | 4-hexenoic acid | 0 | 1 |
| Pest-B | Compound-F | 4-hexenoic acid | 0 | 1 |
| Pest-B | Compound-A | 4-methylhexanoic acid | 0 | 1 |
| Pest-A | Compound-A | 4-methylhexanoic acid | 1 | 4 |
| Pest-C | Compound-B | 4-methylhexanoic acid | 0 | 1 |
| Pest-B | Compound-B | 4-methylhexanoic acid | 0 | 1 |
| Pest-A | Compound-B | 4-methylhexanoic acid | 1 | 4 |
| Pest-C | Compound-C | 4-methylhexanoic acid | 0 | 1 |
| Pest-C | Compound-D | 4-methylhexanoic acid | 0 | 1 |
| Pest-B | Compound-D | 4-methylhexanoic acid | 0 | 1 |
| Pest-C | Compound-E | 4-methylhexanoic acid | 0 | 1 |
| Pest-A | Compound-E | 4-methylhexanoic acid | 1 | 4 |
| Pest-B | Compound-E | 4-methylhexanoic acid | 1 | 4 |
| Pest-C | Compound-F | 4-methylhexanoic acid | 0 | 1 |
| Pest-B | Compound-F | 4-methylhexanoic acid | 0 | 1 |
| Pest-A | Compound-F | 4-methylhexanoic acid | 1 | 4 |
| Pest-C | Compound-A | 5-hexenoic acid | 0 | 1 |
| Pest-A | Compound-A | 5-hexenoic acid | 1 | 2 |
| Pest-B | Compound-A | 5-hexenoic acid | 1 | 6 |
| Pest-A | Compound-G | 5-hexenoic acid | 0 | 1 |
| Pest-C | Compound-G | 5-hexenoic acid | 0 | 1 |
| Pest-B | Compound-G | 5-hexenoic acid | 0 | 1 |
| Pest-C | Compound-B | 5-hexenoic acid | 0 | 1 |
| Pest-B | Compound-B | 5-hexenoic acid | 0 | 1 |
| Pest-A | Compound-B | 5-hexenoic acid | 1 | 2 |
| Pest-A | Compound-C | 5-hexenoic acid | 0 | 1 |
| Pest-C | Compound-C | 5-hexenoic acid | 0 | 1 |
| Pest-B | Compound-C | 5-hexenoic acid | 0 | 1 |
| Pest-A | Compound-D | 5-hexenoic acid | 0 | 1 |
| Pest-C | Compound-D | 5-hexenoic acid | 0 | 1 |
| Pest-B | Compound-D | 5-hexenoic acid | 0 | 1 |
| Pest-A | Compound-H | 5-hexenoic acid | 0 | 1 |
| Pest-A | Compound-I | 5-hexenoic acid | 0 | 1 |
| Pest-C | Compound-I | 5-hexenoic acid | 0 | 1 |
| Pest-B | Compound-I | 5-hexenoic acid | 0 | 1 |
| Pest-A | Compound-E | 5-hexenoic acid | 1 | 4 |
| Pest-C | Compound-E | 5-hexenoic acid | 1 | 4 |
| Pest-B | Compound-E | 5-hexenoic acid | 1 | 8 |
| Pest-C | Compound-F | 5-hexenoic acid | 0 | 1 |
| Pest-B | Compound-F | 5-hexenoic acid | 0 | 1 |
| Pest-A | Compound-F | 5-hexenoic acid | 1 | 4 |
| Pest-C | Compound-A | 7-octenoic acid | 0 | 1 |
| Pest-A | Compound-A | 7-octenoic acid | 1 | 4 |
| Pest-A | Compound-G | 7-octenoic acid | 0 | 1 |
| Pest-C | Compound-G | 7-octenoic acid | 0 | 1 |
| Pest-B | Compound-G | 7-octenoic acid | 0 | 1 |
| Pest-A | Compound-B | 7-octenoic acid | 0 | 1 |
| Pest-B | Compound-B | 7-octenoic acid | 0 | 1 |
| Pest-C | Compound-B | 7-octenoic acid | 1 | 4 |
| Pest-A | Compound-C | 7-octenoic acid | 0 | 1 |
| Pest-C | Compound-C | 7-octenoic acid | 0 | 1 |
| Pest-B | Compound-C | 7-octenoic acid | 0 | 1 |
| Pest-A | Compound-D | 7-octenoic acid | 0 | 1 |
| Pest-C | Compound-D | 7-octenoic acid | 0 | 1 |
| Pest-B | Compound-D | 7-octenoic acid | 0 | 1 |
| Pest-A | Compound-H | 7-octenoic acid | 0 | 1 |
| Pest-A | Compound-I | 7-octenoic acid | 0 | 1 |
| Pest-C | Compound-I | 7-octenoic acid | 0 | 1 |
| Pest-B | Compound-I | 7-octenoic acid | 0 | 1 |
| Pest-A | Compound-E | 7-octenoic acid | 1 | 6 |
| Pest-C | Compound-E | 7-octenoic acid | 1 | 8 |
| Pest-B | Compound-E | 7-octenoic acid | 1 | 16 |
| Pest-A | Compound-F | 7-octenoic acid | 0 | 1 |
| Pest-B | Compound-F | 7-octenoic acid | 0 | 1 |
| Pest-C | Compound-F | 7-octenoic acid | 1 | 4 |
| Pest-B | Compound-A | 8-hydroxyoctanoic acid | 1 | 4 |
| Pest-C | Compound-B | 8-hydroxyoctanoic acid | 0 | 1 |
| Pest-B | Compound-B | 8-hydroxyoctanoic acid | 0 | 1 |
| Pest-A | Compound-B | 8-hydroxyoctanoic acid | 1 | 4 |
| Pest-A | Compound-D | 8-hydroxyoctanoic acid | 1 | 4 |
| Pest-C | Compound-E | 8-hydroxyoctanoic acid | 0 | 1 |
| Pest-A | Compound-E | 8-hydroxyoctanoic acid | 1 | 4 |
| Pest-B | Compound-E | 8-hydroxyoctanoic acid | 1 | 4 |
| Pest-C | Compound-F | 8-hydroxyoctanoic acid | 0 | 1 |
| Pest-B | Compound-F | 8-hydroxyoctanoic acid | 0 | 1 |
| Pest-A | Compound-F | 8-hydroxyoctanoic acid | 1 | 4 |
| Pest-A | Compound-A | 9-decenoic acid | 1 | 4 |
| Pest-C | Compound-A | 9-decenoic acid | 1 | 4 |
| Pest-B | Compound-A | 9-decenoic acid | 1 | 12 |
| Pest-C | Compound-G | 9-decenoic acid | 0 | 1 |
| Pest-A | Compound-G | 9-decenoic acid | 1 | 4 |
| Pest-B | Compound-G | 9-decenoic acid | 1 | 4 |
| Pest-A | Compound-B | 9-decenoic acid | 0 | 1 |
| Pest-C | Compound-B | 9-decenoic acid | 1 | 4 |
| Pest-B | Compound-B | 9-decenoic acid | 1 | 4 |
| Pest-C | Compound-C | 9-decenoic acid | 0 | 1 |
| Pest-A | Compound-C | 9-decenoic acid | 1 | 8 |
| Pest-B | Compound-C | 9-decenoic acid | 1 | 4 |
| Pest-A | Compound-D | 9-decenoic acid | 0 | 1 |
| Pest-C | Compound-D | 9-decenoic acid | 0 | 1 |
| Pest-B | Compound-D | 9-decenoic acid | 1 | 2 |
| Pest-A | Compound-H | 9-decenoic acid | 1 | 4 |
| Pest-A | Compound-I | 9-decenoic acid | 0 | 1 |
| Pest-C | Compound-I | 9-decenoic acid | 0 | 1 |
| Pest-B | Compound-I | 9-decenoic acid | 1 | 8 |
| Pest-A | Compound-E | 9-decenoic acid | 1 | 8 |
| Pest-C | Compound-E | 9-decenoic acid | 1 | 4 |
| Pest-B | Compound-E | 9-decenoic acid | 1 | 16 |
| Pest-A | Compound-F | 9-decenoic acid | 1 | 8 |
| Pest-C | Compound-F | 9-decenoic acid | 1 | 4 |
| Pest-B | Compound-F | 9-decenoic acid | 1 | 4 |
| Pest-C | Compound-A | decanoic acid | 0 | 1 |
| Pest-A | Compound-A | decanoic acid | 1 | 8 |
| Pest-B | Compound-A | decanoic acid | 1 | 4 |
| Pest-A | Compound-G | decanoic acid | 0 | 1 |
| Pest-C | Compound-G | decanoic acid | 0 | 1 |
| Pest-B | Compound-G | decanoic acid | 0 | 1 |
| Pest-A | Compound-B | decanoic acid | 1 | 8 |
| Pest-C | Compound-B | decanoic acid | 1 | 2 |
| Pest-B | Compound-B | decanoic acid | 1 | 2 |
| Pest-C | Compound-C | decanoic acid | 0 | 1 |
| Pest-A | Compound-C | decanoic acid | 1 | 8 |
| Pest-B | Compound-C | decanoic acid | 1 | 4 |
| Pest-A | Compound-D | decanoic acid | 0 | 1 |
| Pest-C | Compound-D | decanoic acid | 0 | 1 |
| Pest-B | Compound-D | decanoic acid | 0 | 1 |
| Pest-A | Compound-H | decanoic acid | 1 | 8 |
| Pest-C | Compound-I | decanoic acid | 0 | 1 |
| Pest-B | Compound-I | decanoic acid | 0 | 1 |
| Pest-A | Compound-I | decanoic acid | 1 | 8 |
| Pest-A | Compound-E | decanoic acid | 1 | 16 |
| Pest-C | Compound-E | decanoic acid | 1 | 4 |
| Pest-B | Compound-E | decanoic acid | 1 | 8 |
| Pest-A | Compound-F | decanoic acid | 1 | 16 |
| Pest-C | Compound-F | decanoic acid | 1 | 4 |
| Pest-B | Compound-F | decanoic acid | 1 | 4 |
| Pest-A | Compound-A | dodecanoic acid | 0 | 1 |
| Pest-C | Compound-A | dodecanoic acid | 1 | 4 |
| Pest-B | Compound-A | dodecanoic acid | 1 | 16 |
| Pest-A | Compound-G | dodecanoic acid | 0 | 1 |
| Pest-B | Compound-G | dodecanoic acid | 0 | 1 |
| Pest-C | Compound-G | dodecanoic acid | 1 | 2 |
| Pest-C | Compound-B | dodecanoic acid | 0 | 1 |
| Pest-A | Compound-B | dodecanoic acid | 1 | 4 |
| Pest-B | Compound-B | dodecanoic acid | 1 | 4 |
| Pest-A | Compound-C | dodecanoic acid | 0 | 1 |
| Pest-C | Compound-C | dodecanoic acid | 0 | 1 |
| Pest-B | Compound-C | dodecanoic acid | 1 | 8 |
| Pest-A | Compound-D | dodecanoic acid | 0 | 1 |
| Pest-B | Compound-D | dodecanoic acid | 0 | 1 |
| Pest-C | Compound-D | dodecanoic acid | 1 | 4 |
| Pest-A | Compound-H | dodecanoic acid | 1 | 4 |
| Pest-C | Compound-I | dodecanoic acid | 0 | 1 |
| Pest-A | Compound-I | dodecanoic acid | 1 | 4 |
| Pest-B | Compound-I | dodecanoic acid | 1 | 4 |
| Pest-A | Compound-E | dodecanoic acid | 1 | 2 |
| Pest-C | Compound-E | dodecanoic acid | 1 | 4 |
| Pest-B | Compound-E | dodecanoic acid | 1 | 8 |
| Pest-A | Compound-F | dodecanoic acid | 1 | 8 |
| Pest-C | Compound-F | dodecanoic acid | 1 | 4 |
| Pest-B | Compound-F | dodecanoic acid | 1 | 8 |
| Pest-A | Compound-A | heptanoic acid | 1 | 2 |
| Pest-C | Compound-A | heptanoic acid | 1 | 4 |
| Pest-B | Compound-A | heptanoic acid | 1 | 4 |
| Pest-A | Compound-G | heptanoic acid | 0 | 1 |
| Pest-B | Compound-G | heptanoic acid | 0 | 1 |
| Pest-C | Compound-G | heptanoic acid | 1 | 2 |
| Pest-B | Compound-B | heptanoic acid | 0 | 1 |
| Pest-A | Compound-B | heptanoic acid | 1 | 4 |
| Pest-C | Compound-B | heptanoic acid | 1 | 4 |
| Pest-A | Compound-C | heptanoic acid | 0 | 1 |
| Pest-C | Compound-C | heptanoic acid | 0 | 1 |
| Pest-B | Compound-C | heptanoic acid | 1 | 4 |
| Pest-A | Compound-D | heptanoic acid | 0 | 1 |
| Pest-C | Compound-D | heptanoic acid | 0 | 1 |
| Pest-B | Compound-D | heptanoic acid | 0 | 1 |
| Pest-A | Compound-H | heptanoic acid | 0 | 1 |
| Pest-A | Compound-I | heptanoic acid | 0 | 1 |
| Pest-C | Compound-I | heptanoic acid | 0 | 1 |
| Pest-B | Compound-I | heptanoic acid | 0 | 1 |
| Pest-A | Compound-E | heptanoic acid | 1 | 2 |
| Pest-C | Compound-E | heptanoic acid | 1 | 4 |
| Pest-B | Compound-E | heptanoic acid | 1 | 16 |
| Pest-A | Compound-F | heptanoic acid | 1 | 8 |
| Pest-C | Compound-F | heptanoic acid | 1 | 4 |
| Pest-B | Compound-F | heptanoic acid | 1 | 8 |
| Pest-A | Compound-A | hexanoic acid | 1 | 2 |
| Pest-C | Compound-A | hexanoic acid | 1 | 4 |
| Pest-B | Compound-A | hexanoic acid | 1 | 4 |
| Pest-A | Compound-G | hexanoic acid | 0 | 1 |
| Pest-C | Compound-G | hexanoic acid | 0 | 1 |
| Pest-B | Compound-G | hexanoic acid | 0 | 1 |
| Pest-C | Compound-B | hexanoic acid | 0 | 1 |
| Pest-B | Compound-B | hexanoic acid | 0 | 1 |
| Pest-A | Compound-B | hexanoic acid | 1 | 4 |
| Pest-A | Compound-C | hexanoic acid | 0 | 1 |
| Pest-C | Compound-C | hexanoic acid | 0 | 1 |
| Pest-B | Compound-C | hexanoic acid | 0 | 1 |
| Pest-A | Compound-D | hexanoic acid | 0 | 1 |
| Pest-C | Compound-D | hexanoic acid | 0 | 1 |
| Pest-B | Compound-D | hexanoic acid | 0 | 1 |
| Pest-A | Compound-H | hexanoic acid | 0 | 1 |
| Pest-A | Compound-I | hexanoic acid | 0 | 1 |
| Pest-C | Compound-I | hexanoic acid | 0 | 1 |
| Pest-B | Compound-I | hexanoic acid | 0 | 1 |
| Pest-A | Compound-E | hexanoic acid | 1 | 2 |
| Pest-C | Compound-E | hexanoic acid | 1 | 8 |
| Pest-B | Compound-E | hexanoic acid | 1 | 8 |
| Pest-C | Compound-F | hexanoic acid | 0 | 1 |
| Pest-A | Compound-F | hexanoic acid | 1 | 8 |
| Pest-B | Compound-F | hexanoic acid | 1 | 8 |
| Pest-A | Compound-A | nonanoic acid | 1 | 2 |
| Pest-C | Compound-A | nonanoic acid | 1 | 4 |
| Pest-B | Compound-A | nonanoic acid | 1 | 8 |
| Pest-A | Compound-G | nonanoic acid | 0 | 1 |
| Pest-B | Compound-G | nonanoic acid | 0 | 1 |
| Pest-C | Compound-G | nonanoic acid | 1 | 2 |
| Pest-C | Compound-B | nonanoic acid | 0 | 1 |
| Pest-A | Compound-B | nonanoic acid | 1 | 4 |
| Pest-B | Compound-B | nonanoic acid | 1 | 2 |
| Pest-A | Compound-C | nonanoic acid | 0 | 1 |
| Pest-C | Compound-C | nonanoic acid | 0 | 1 |
| Pest-B | Compound-C | nonanoic acid | 0 | 1 |
| Pest-A | Compound-D | nonanoic acid | 0 | 1 |
| Pest-B | Compound-D | nonanoic acid | 0 | 1 |
| Pest-C | Compound-D | nonanoic acid | 1 | 4 |
| Pest-A | Compound-H | nonanoic acid | 0 | 1 |
| Pest-A | Compound-I | nonanoic acid | 0 | 1 |
| Pest-C | Compound-I | nonanoic acid | 0 | 1 |
| Pest-B | Compound-I | nonanoic acid | 0 | 1 |
| Pest-A | Compound-E | nonanoic acid | 1 | 4 |
| Pest-C | Compound-E | nonanoic acid | 1 | 4 |
| Pest-B | Compound-E | nonanoic acid | 1 | 16 |
| Pest-A | Compound-F | nonanoic acid | 1 | 8 |
| Pest-C | Compound-F | nonanoic acid | 1 | 4 |
| Pest-B | Compound-F | nonanoic acid | 1 | 8 |
| Pest-A | Compound-A | octanoic acid | 1 | 2 |
| Pest-C | Compound-A | octanoic acid | 1 | 4 |
| Pest-B | Compound-A | octanoic acid | 1 | 8 |
| Pest-A | Compound-G | octanoic acid | 0 | 1 |
| Pest-B | Compound-G | octanoic acid | 0 | 1 |
| Pest-C | Compound-G | octanoic acid | 1 | 2 |
| Pest-B | Compound-B | octanoic acid | 0 | 1 |
| Pest-A | Compound-B | octanoic acid | 1 | 4 |
| Pest-C | Compound-B | octanoic acid | 1 | 4 |
| Pest-A | Compound-C | octanoic acid | 0 | 1 |
| Pest-C | Compound-C | octanoic acid | 0 | 1 |
| Pest-B | Compound-C | octanoic acid | 1 | 8 |
| Pest-A | Compound-D | octanoic acid | 0 | 1 |
| Pest-C | Compound-D | octanoic acid | 0 | 1 |
| Pest-B | Compound-D | octanoic acid | 0 | 1 |
| Pest-A | Compound-H | octanoic acid | 1 | 16 |
| Pest-A | Compound-I | octanoic acid | 0 | 1 |
| Pest-C | Compound-I | octanoic acid | 0 | 1 |
| Pest-B | Compound-I | octanoic acid | 0 | 1 |
| Pest-A | Compound-E | octanoic acid | 1 | 4 |
| Pest-C | Compound-E | octanoic acid | 1 | 8 |
| Pest-B | Compound-E | octanoic acid | 1 | 8 |
| Pest-A | Compound-F | octanoic acid | 1 | 8 |
| Pest-C | Compound-F | octanoic acid | 1 | 4 |
| Pest-B | Compound-F | octanoic acid | 1 | 8 |
| Pest-C | Compound-A | oleic acid | 0 | 1 |
| Pest-A | Compound-A | oleic acid | 1 | 2 |
| Pest-A | Compound-G | oleic acid | 0 | 1 |
| Pest-C | Compound-G | oleic acid | 0 | 1 |
| Pest-B | Compound-G | oleic acid | 0 | 1 |
| Pest-A | Compound-B | oleic acid | 0 | 1 |
| Pest-C | Compound-B | oleic acid | 0 | 1 |
| Pest-B | Compound-B | oleic acid | 0 | 1 |
| Pest-A | Compound-C | oleic acid | 0 | 1 |
| Pest-C | Compound-C | oleic acid | 0 | 1 |
| Pest-B | Compound-C | oleic acid | 0 | 1 |
| Pest-A | Compound-D | oleic acid | 0 | 1 |
| Pest-C | Compound-D | oleic acid | 0 | 1 |
| Pest-B | Compound-D | oleic acid | 0 | 1 |
| Pest-A | Compound-H | oleic acid | 0 | 1 |
| Pest-A | Compound-I | oleic acid | 0 | 1 |
| Pest-C | Compound-I | oleic acid | 0 | 1 |
| Pest-B | Compound-I | oleic acid | 0 | 1 |
| Pest-C | Compound-E | oleic acid | 0 | 1 |
| Pest-B | Compound-E | oleic acid | 0 | 1 |
| Pest-A | Compound-F | oleic acid | 0 | 1 |
| Pest-C | Compound-F | oleic acid | 0 | 1 |
| Pest-B | Compound-F | oleic acid | 0 | 1 |
| Pest-A | Compound-A | sorbic acid | 0 | 1 |
| Pest-C | Compound-A | sorbic acid | 0 | 1 |
| Pest-B | Compound-A | sorbic acid | 0 | 1 |
| Pest-A | Compound-G | sorbic acid | 0 | 1 |
| Pest-C | Compound-G | sorbic acid | 0 | 1 |
| Pest-B | Compound-G | sorbic acid | 0 | 1 |
| Pest-A | Compound-B | sorbic acid | 0 | 1 |
| Pest-C | Compound-B | sorbic acid | 0 | 1 |
| Pest-B | Compound-B | sorbic acid | 0 | 1 |
| Pest-A | Compound-C | sorbic acid | 0 | 1 |
| Pest-C | Compound-C | sorbic acid | 0 | 1 |
| Pest-B | Compound-C | sorbic acid | 0 | 1 |
| Pest-A | Compound-D | sorbic acid | 0 | 1 |
| Pest-C | Compound-D | sorbic acid | 0 | 1 |
| Pest-B | Compound-D | sorbic acid | 0 | 1 |
| Pest-A | Compound-H | sorbic acid | 0 | 1 |
| Pest-A | Compound-I | sorbic acid | 0 | 1 |
| Pest-C | Compound-I | sorbic acid | 0 | 1 |
| Pest-B | Compound-I | sorbic acid | 0 | 1 |
| Pest-A | Compound-E | sorbic acid | 1 | 6 |
| Pest-C | Compound-E | sorbic acid | 1 | 4 |
| Pest-B | Compound-E | sorbic acid | 1 | 4 |
| Pest-A | Compound-F | sorbic acid | 0 | 1 |
| Pest-C | Compound-F | sorbic acid | 0 | 1 |
| Pest-B | Compound-F | sorbic acid | 0 | 1 |
| Pest-C | Compound-A | trans-2-decenoic acid | 0 | 1 |
| Pest-A | Compound-A | trans-2-decenoic acid | 1 | 8 |
| Pest-B | Compound-A | trans-2-decenoic acid | 1 | 4 |
| Pest-A | Compound-G | trans-2-decenoic acid | 0 | 1 |
| Pest-C | Compound-G | trans-2-decenoic acid | 0 | 1 |
| Pest-B | Compound-G | trans-2-decenoic acid | 0 | 1 |
| Pest-A | Compound-B | trans-2-decenoic acid | 0 | 1 |
| Pest-B | Compound-B | trans-2-decenoic acid | 1 | 4 |
| Pest-C | Compound-C | trans-2-decenoic acid | 0 | 1 |
| Pest-A | Compound-C | trans-2-decenoic acid | 1 | 8 |
| Pest-B | Compound-C | trans-2-decenoic acid | 1 | 4 |
| Pest-A | Compound-D | trans-2-decenoic acid | 0 | 1 |
| Pest-C | Compound-D | trans-2-decenoic acid | 0 | 1 |
| Pest-B | Compound-D | trans-2-decenoic acid | 1 | 2 |
| Pest-A | Compound-H | trans-2-decenoic acid | 0 | 1 |
| Pest-A | Compound-I | trans-2-decenoic acid | 0 | 1 |
| Pest-C | Compound-I | trans-2-decenoic acid | 0 | 1 |
| Pest-B | Compound-I | trans-2-decenoic acid | 1 | 4 |
| Pest-C | Compound-E | trans-2-decenoic acid | 0 | 1 |
| Pest-A | Compound-E | trans-2-decenoic acid | 1 | 6 |
| Pest-B | Compound-E | trans-2-decenoic acid | 1 | 8 |
| Pest-A | Compound-F | trans-2-decenoic acid | 1 | 4 |
| Pest-C | Compound-F | trans-2-decenoic acid | 1 | 4 |
| Pest-B | Compound-F | trans-2-decenoic acid | 1 | 4 |
| Pest-C | Compound-A | trans-2-hexenoic acid | 0 | 1 |
| Pest-A | Compound-A | trans-2-hexenoic acid | 1 | 2 |
| Pest-A | Compound-G | trans-2-hexenoic acid | 0 | 1 |
| Pest-C | Compound-G | trans-2-hexenoic acid | 0 | 1 |
| Pest-B | Compound-G | trans-2-hexenoic acid | 0 | 1 |
| Pest-A | Compound-B | trans-2-hexenoic acid | 0 | 1 |
| Pest-C | Compound-B | trans-2-hexenoic acid | 0 | 1 |
| Pest-B | Compound-B | trans-2-hexenoic acid | 0 | 1 |
| Pest-A | Compound-C | trans-2-hexenoic acid | 0 | 1 |
| Pest-C | Compound-C | trans-2-hexenoic acid | 0 | 1 |
| Pest-B | Compound-C | trans-2-hexenoic acid | 0 | 1 |
| Pest-A | Compound-D | trans-2-hexenoic acid | 0 | 1 |
| Pest-C | Compound-D | trans-2-hexenoic acid | 0 | 1 |
| Pest-B | Compound-D | trans-2-hexenoic acid | 0 | 1 |
| Pest-A | Compound-H | trans-2-hexenoic acid | 0 | 1 |
| Pest-A | Compound-I | trans-2-hexenoic acid | 0 | 1 |
| Pest-C | Compound-I | trans-2-hexenoic acid | 0 | 1 |
| Pest-B | Compound-I | trans-2-hexenoic acid | 0 | 1 |
| Pest-C | Compound-E | trans-2-hexenoic acid | 0 | 1 |
| Pest-A | Compound-E | trans-2-hexenoic acid | 1 | 5 |
| Pest-B | Compound-E | trans-2-hexenoic acid | 1 | 8 |
| Pest-A | Compound-F | trans-2-hexenoic acid | 0 | 1 |
| Pest-C | Compound-F | trans-2-hexenoic acid | 0 | 1 |
| Pest-B | Compound-F | trans-2-hexenoic acid | 0 | 1 |
| Pest-A | Compound-A | trans-2-nonenoic acid | 1 | 4 |
| Pest-C | Compound-A | trans-2-nonenoic acid | 1 | 4 |
| Pest-B | Compound-A | trans-2-nonenoic acid | 1 | 16 |
| Pest-C | Compound-G | trans-2-nonenoic acid | 0 | 1 |
| Pest-A | Compound-G | trans-2-nonenoic acid | 1 | 4 |
| Pest-B | Compound-G | trans-2-nonenoic acid | 1 | 8 |
| Pest-A | Compound-B | trans-2-nonenoic acid | 0 | 1 |
| Pest-C | Compound-B | trans-2-nonenoic acid | 1 | 4 |
| Pest-B | Compound-B | trans-2-nonenoic acid | 1 | 4 |
| Pest-C | Compound-C | trans-2-nonenoic acid | 0 | 1 |
| Pest-A | Compound-C | trans-2-nonenoic acid | 1 | 8 |
| Pest-B | Compound-C | trans-2-nonenoic acid | 1 | 4 |
| Pest-A | Compound-D | trans-2-nonenoic acid | 0 | 1 |
| Pest-C | Compound-D | trans-2-nonenoic acid | 0 | 1 |
| Pest-B | Compound-D | trans-2-nonenoic acid | 0 | 1 |
| Pest-A | Compound-H | trans-2-nonenoic acid | 0 | 1 |
| Pest-A | Compound-I | trans-2-nonenoic acid | 0 | 1 |
| Pest-C | Compound-I | trans-2-nonenoic acid | 0 | 1 |
| Pest-B | Compound-I | trans-2-nonenoic acid | 1 | 4 |
| Pest-C | Compound-E | trans-2-nonenoic acid | 0 | 1 |
| Pest-A | Compound-E | trans-2-nonenoic acid | 1 | 6 |
| Pest-B | Compound-E | trans-2-nonenoic acid | 1 | 16 |
| Pest-A | Compound-F | trans-2-nonenoic acid | 0 | 1 |
| Pest-C | Compound-F | trans-2-nonenoic acid | 1 | 8 |
| Pest-B | Compound-F | trans-2-nonenoic acid | 1 | 4 |
| Pest-C | Compound-A | trans-2-octenoic acid | 0 | 1 |
| Pest-A | Compound-A | trans-2-octenoic acid | 1 | 8 |
| Pest-A | Compound-G | trans-2-octenoic acid | 0 | 1 |
| Pest-C | Compound-G | trans-2-octenoic acid | 0 | 1 |
| Pest-B | Compound-G | trans-2-octenoic acid | 0 | 1 |
| Pest-A | Compound-B | trans-2-octenoic acid | 0 | 1 |
| Pest-C | Compound-B | trans-2-octenoic acid | 1 | 4 |
| Pest-B | Compound-B | trans-2-octenoic acid | 1 | 4 |
| Pest-B | Compound-C | trans-2-octenoic acid | 0 | 1 |
| Pest-A | Compound-C | trans-2-octenoic acid | 1 | 4 |
| Pest-C | Compound-C | trans-2-octenoic acid | 1 | 4 |
| Pest-A | Compound-D | trans-2-octenoic acid | 0 | 1 |
| Pest-C | Compound-D | trans-2-octenoic acid | 0 | 1 |
| Pest-B | Compound-D | trans-2-octenoic acid | 1 | 2 |
| Pest-A | Compound-H | trans-2-octenoic acid | 0 | 1 |
| Pest-A | Compound-I | trans-2-octenoic acid | 0 | 1 |
| Pest-C | Compound-I | trans-2-octenoic acid | 0 | 1 |
| Pest-B | Compound-I | trans-2-octenoic acid | 0 | 1 |
| Pest-A | Compound-E | trans-2-octenoic acid | 1 | 8 |
| Pest-C | Compound-E | trans-2-octenoic acid | 1 | 8 |
| Pest-B | Compound-E | trans-2-octenoic acid | 1 | 16 |
| Pest-B | Compound-F | trans-2-octenoic acid | 0 | 1 |
| Pest-A | Compound-F | trans-2-octenoic acid | 1 | 8 |
| Pest-C | Compound-F | trans-2-octenoic acid | 1 | 4 |
| Pest-C | Compound-A | trans-2-undecenoic acid | 0 | 1 |
| Pest-A | Compound-A | trans-2-undecenoic acid | 1 | 4 |
| Pest-B | Compound-A | trans-2-undecenoic acid | 1 | 8 |
| Pest-A | Compound-G | trans-2-undecenoic acid | 0 | 1 |
| Pest-C | Compound-G | trans-2-undecenoic acid | 0 | 1 |
| Pest-B | Compound-G | trans-2-undecenoic acid | 0 | 1 |
| Pest-A | Compound-B | trans-2-undecenoic acid | 0 | 1 |
| Pest-B | Compound-B | trans-2-undecenoic acid | 1 | 4 |
| Pest-C | Compound-C | trans-2-undecenoic acid | 0 | 1 |
| Pest-B | Compound-C | trans-2-undecenoic acid | 0 | 1 |
| Pest-A | Compound-C | trans-2-undecenoic acid | 1 | 4 |
| Pest-A | Compound-D | trans-2-undecenoic acid | 0 | 1 |
| Pest-C | Compound-D | trans-2-undecenoic acid | 0 | 1 |
| Pest-B | Compound-D | trans-2-undecenoic acid | 0 | 1 |
| Pest-A | Compound-H | trans-2-undecenoic acid | 1 | 4 |
| Pest-A | Compound-I | trans-2-undecenoic acid | 0 | 1 |
| Pest-C | Compound-I | trans-2-undecenoic acid | 0 | 1 |
| Pest-B | Compound-I | trans-2-undecenoic acid | 1 | 4 |
| Pest-C | Compound-E | trans-2-undecenoic acid | 0 | 1 |
| Pest-A | Compound-E | trans-2-undecenoic acid | 1 | 5 |
| Pest-B | Compound-E | trans-2-undecenoic acid | 1 | 16 |
| Pest-A | Compound-F | trans-2-undecenoic acid | 1 | 4 |
| Pest-C | Compound-F | trans-2-undecenoic acid | 1 | 4 |
| Pest-B | Compound-F | trans-2-undecenoic acid | 1 | 2 |
| Pest-A | Compound-A | trans-3-hexenoic acid | 1 | 2 |
| Pest-C | Compound-A | trans-3-hexenoic acid | 1 | 4 |
| Pest-B | Compound-A | trans-3-hexenoic acid | 1 | 4 |
| Pest-A | Compound-G | trans-3-hexenoic acid | 0 | 1 |
| Pest-C | Compound-G | trans-3-hexenoic acid | 0 | 1 |
| Pest-B | Compound-G | trans-3-hexenoic acid | 0 | 1 |
| Pest-A | Compound-B | trans-3-hexenoic acid | 0 | 1 |
| Pest-B | Compound-B | trans-3-hexenoic acid | 0 | 1 |
| Pest-A | Compound-C | trans-3-hexenoic acid | 0 | 1 |
| Pest-B | Compound-C | trans-3-hexenoic acid | 0 | 1 |
| Pest-C | Compound-C | trans-3-hexenoic acid | 1 | 4 |
| Pest-A | Compound-D | trans-3-hexenoic acid | 0 | 1 |
| Pest-C | Compound-D | trans-3-hexenoic acid | 0 | 1 |
| Pest-B | Compound-D | trans-3-hexenoic acid | 0 | 1 |
| Pest-A | Compound-H | trans-3-hexenoic acid | 0 | 1 |
| Pest-A | Compound-I | trans-3-hexenoic acid | 0 | 1 |
| Pest-C | Compound-I | trans-3-hexenoic acid | 0 | 1 |
| Pest-B | Compound-I | trans-3-hexenoic acid | 0 | 1 |
| Pest-A | Compound-E | trans-3-hexenoic acid | 1 | 4 |
| Pest-C | Compound-E | trans-3-hexenoic acid | 1 | 8 |
| Pest-B | Compound-E | trans-3-hexenoic acid | 1 | 8 |
| Pest-A | Compound-F | trans-3-hexenoic acid | 0 | 1 |
| Pest-C | Compound-F | trans-3-hexenoic acid | 0 | 1 |
| Pest-B | Compound-F | trans-3-hexenoic acid | 1 | 2 |

Overall, the results of these tests suggest that, in at least some circumstances, the herein-described systems and methods are comparable in predictive accuracy to an experienced human chemist.

### CONCLUSIONS

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method for generating a prediction of a synergistic interaction between two or more compounds against one or more pests, the method performed by one or more processors and comprising:
receiving a first representation of a pesticidal compound;
receiving a second representation of a synergistic compound;
generating an encoded representation of a composition comprising the pesticidal and synergistic compounds by encoding a first chemical feature of the pesticidal compound and a second chemical feature of the synergistic compound based on the respective first and second representations; and
generating one or more predictions of a synergistic interaction between the pesticidal compound and the synergistic compound against one or more pests, said generating comprising:
transforming the encoded representation based on trained parameters of a classifier, the trained parameters of the classifier having been trained over at least one synergistic interaction between compounds of at least one composition against at least one training pest.

2. The method according to claim 1 wherein the one or more predictions of synergistic interaction comprise a plurality of predictions and the method further comprises: combining the plurality of synergy predictions into a combined synergy.

3. The method according to claim 2 wherein the method further comprises determining at least one of: a confidence interval, a standard deviation, and a variance based on the plurality of predictions.

4. The method according to claim 3 wherein the classifier comprises a stochastic classifier, generating the one or more predictions comprises transforming the encoded representation based on the trained parameters of the classifier over a plurality of iterations and generating a prediction for each iteration.

5. The method according to claim 1 wherein generating the encoded representation comprises generating a first encoded compound representation based on the first chemical feature of the pesticidal compound and generating a second encoded compound representation based on the second chemical feature of the synergistic compound and wherein generating the one or more predictions comprises generating the one or more predictions based on the first and second encoded compound representations.

6. The method according to claim 1 wherein generating the encoded representation comprises generating the encoded representation to be lower-dimensional than at least one of the first and second representations;
optionally wherein the trained parameters of the encoder model have been trained over a different training set than the trained parameters of the classifier.

7. The method according to claim 1 wherein the generating the encoded representation comprises transforming the first and second chemical features of the respective pesticidal and synergistic compounds into the encoded representation based on trained parameters of an encoder model;
optionally wherein the encoder model comprises an encoder portion of a variational autoencoder, the encoder portion operable to transform the first and second chemical features from an input space to a latent space of the variational autoencoder.

8. (canceled)

9. (canceled)

10. The method according to claim 1 further comprising selecting the classifier from a plurality of classifiers based on the one or more pests, the method optionally further comprising receiving a representation of the one or more pests and selecting the classifier comprises selecting the classifier based on the representation of the one or more pests.

11. (canceled)

12. The method according to claim 10 wherein the classifier is a first one of a plurality of classifiers, at least a second classifier of the plurality having been trained against different pests than the one or more pests, and selecting the classifier from the plurality of classifiers comprises selecting one of the first and second classifiers based on the one or more pests.

13. The method according to claim 10 wherein the classifier comprises an ensemble classifier comprising a plurality of constituent classifiers, the plurality of constituent classifiers comprising at least a first constituent classifier and a second constituent classifier, respective trained parameters of the first and second constituent classifiers each having been trained over at least one synergistic interaction between compounds of at least one composition against at least one of the one or more pests;
optionally wherein generating one or more predictions comprises generating a first prediction based on the first constituent classifier and generating a second prediction based on the second constituent classifier.

14. (canceled)

15. The method according to claim 1 comprising generating an enhanced representation of at least one of the pesticidal and synergistic compounds, the enhanced representation comprising an enhanced chemical feature of the at least one of the pesticidal and synergistic compounds, the enhanced chemical feature not contained by the first and second representations;
optionally wherein generating the enhanced representation comprises determining the enhanced chemical feature based on trained parameters of a quantitative structure-activity relationship model.

16. (canceled)

17. The method according to claim 1 comprising receiving a third representation of a third compound and excluding an excluded composition comprising the third compound from prediction based on determining at least one of: a chemical feature of the third compound matches an exclusion rule, an availability value corresponding to the third compound being less than a threshold, a similarity metric between the third compound and a fourth compound being greater than a threshold, and a toxicity indication of the third compound matches a toxicity criterion.

18. (canceled)

19. The method according to claim 1 comprising selecting at least one of the first and second chemical features from the group consisting of: representations of aromaticity, representations of electronegativity, representations of polarity, representations of hydrophilicity/hydrophobicity, and representations of hybridizations of at least one of the pesticidal and synergistic compounds.

20. The method according to claim 1 wherein the one or more pests comprise the at least one training pest so that transforming the encoded representation based on trained parameters of a classifier, the trained parameters of the classifier having been trained over at least one synergistic interaction between compounds of at least one composition against at least one training pest comprises transforming the encoded representation based on trained parameters of a classifier, the trained parameters of the classifier having been trained over at least one synergistic interaction between compounds of at least one composition against at least one of the one or more pests.

21. The method according to claim 1 wherein the at least one training pest shares a pesticidal mode of action with at least one of the one or more pests so that transforming the encoded representation based on trained parameters of a classifier, the trained parameters of the classifier having been trained over at least one synergistic interaction between compounds of at least one composition against at least one training pest comprises transforming the encoded representation based on trained parameters of a classifier, the trained parameters of the classifier having been trained over at least one synergistic interaction between compounds of at least one composition against at least one training pest sharing a pesticidal mode of action with at least one of the one or more pests.

22. The method according to claim 1 wherein the trained parameters of the classifier have been trained by:
determining an importance metric for each of a plurality of training compositions;
selecting one or more high-importance compositions from the plurality of training compositions based on the importance metric for each of the one or more high-importance compositions; and
updating the trained parameters of the classified based on the one or more high-importance compositions;
wherein optionally determining the importance metric for a given composition comprises determining the importance metric for the given training composition based on a variance of one or more training predictions of the synergistic interaction between a pesticidal compound of the training composition and a synergistic compound of the training composition.

23. (canceled)

24. The method according to claim 22 wherein selecting one or more high-importance compositions comprises selecting the one or more high-importance compositions based on a representativeness criterion;
wherein selecting the one or more high-importance compositions based on a representativeness criterion comprises determining a plurality of clusters of the plurality of training compositions and selecting at least one high-importance composition from each of at least two of the plurality of clusters;
wherein optionally determining the plurality of clusters of the plurality of training compositions comprises determining a graph similarity metric between at least one graph representing at least one compound of a first one of the training compositions and at least one graph representing at least one compound of a second one of the training compositions.

25. (canceled)

26. (canceled)

27. A computer system comprising:
one or more processors; and
a memory storing instructions which cause the one or more processors to perform operations comprising:
receiving a first representation of a pesticidal compound;
receiving a second representation of a synergistic compound;
generating an encoded representation of a composition comprising the pesticidal and synergistic compounds by encoding a first chemical feature of the pesticidal compound and a second chemical feature of the synergistic compound based on the respective first and second representations; and
generating one or more predictions of a synergistic interaction between the pesticidal compound and the synergistic compound against one or more pests, said generating comprising:
transforming the encoded representation based on trained parameters of a classifier, the trained parameters of the classifier having been trained over at least one synergistic interaction between compounds of at least one composition against at least one training pest.

28. (canceled)

29. A non-transitory machine-readable medium storing instructions which cause one or more processors to perform operations comprising:
receiving a first representation of a pesticidal compound;
receiving a second representation of a synergistic compound;
generating an encoded representation of a composition comprising the pesticidal and synergistic compounds by encoding a first chemical feature of the pesticidal compound and a second chemical feature of the synergistic compound based on the respective first and second representations; and
generating one or more predictions of a synergistic interaction between the pesticidal compound and the synergistic compound against one or more pests, said generating comprising:
transforming the encoded representation based on trained parameters of a classifier, the trained parameters of the classifier having been trained over at least one synergistic interaction between compounds of at least one composition against at least one training pest.

30. (canceled)

31. A method of evaluating a prediction of a synergistic interaction between two or more compounds against one or more pests, the method comprising:
determining a prediction of a synergistic interaction between a pesticidal compound and a synergistic compound by the method of claim 1;
combining the pesticidal compound and the synergistic compound to yield a composition;
exposing the one or more pests to the composition in a test environment; and
evaluating an efficacy of the composition as a pesticide.

32. (canceled)

33. (canceled)

34. (canceled)

35. (canceled)
